(19) United States
(12) Patent Application Publication
(10) Pub. No.: US 2010/0010057 A1
(43) Pub. Date: Jan. 14, 2010

(54) THIAZOLE DERIVATIVES AS INHIBITORS OF P13 KINASE

(75) Inventors: David Charles Festus Moffat, Oxfordshire (GB); Stephen Davies, Oxfordshire (GB); Sonia Maria Alesso, Oxfordshire (GB); Delphine Francoise Monique Launay, Oxfordshire (GB)

Correspondence Address: BANNER & WITCOFF, LTD., 1100 13th STREET, N.W., SUITE 1200, WASHINGTON, DC, 20005-4051, US

(73) Assignee: CHROMA THERAPEUTICS LTD., Oxfordshire (GB)

(21) Appl. No.: 12/299,495

(22) PCT Filed: May 3, 2007
(86) PCT No.: PCT/GB2007/001613

§ 371 Date: May 18, 2009

(30) Foreign Application Priority Data
May 4, 2006 (GB) 0608823.1

(51) Int. Cl. A61K 31/426; C07D 277/38; C12N 9/99; A61P 35/00; A61P 29/00; A61P 27/00

(52) U.S. Cl. 514/370; 548/194; 435/184

(57) ABSTRACT

Compounds of formula (I) are inhibitors of P13 kinase activity, and useful in treatment of, inter alia, autoimmune, inflammatory and proliferative diseases: wherein: s is 0 or 1; U is hydrogen or halogen; X is —(C═O), an optionally substituted divalent phenylene, pyridinylene, pyrimidinylene, or pyrazinylene radical, or a bond; P is optionally substituted C₁-C₆ alkyl and Z is —(CH₂)_{Z}—X₁-L₁-NHCHR₁R₂; or Z is optionally substituted C₁-C₆ alkyl and P is —(CH₂)_{Z}—X₁-L₁-NHCHR₁R₂; R₁ is a carboxylic acid group (—COOH), or an ester group which is hydrolysable by one or more intracellular carboxylesterase enzymes to a carboxylic acid group; R₂ is the side chain of a natural or non-natural alpha amino acid; X₁ is (i) a bond; —NR₄C(═O)NR₅— or —NR₄S(═O)₂—; or except when X is —(C═O)— (ii) —C(═O)—, —S(═O)₂—, or —S(═O)₂NR₄— wherein R₄ and R₅ are independently hydrogen or optionally substituted C₁-C₆ alkyl; and z and L1 are as defined in the specification.

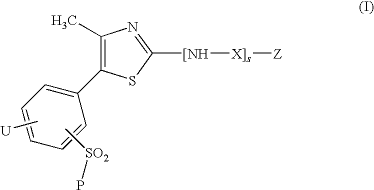

This invention relates to a series of amino acid esters, to compositions containing them, to processes for their preparation and to their use in medicine as PI3 kinase inhibitors for the treatment of autoimmune and inflammatory diseases, including rheumatoid arthritis, psoriasis, inflammatory bowel disease, Crohns disease, ulcerative colitis, chronic obstructive pulmonary disease, asthma, multiple sclerosis, diabetes, atopic dermatitis, graft versus host disease, systemic lupus erythematosus and others. The invention also relates to the use of such compounds in the treatment of proliferative disorders such as cancer, prostate hyperplasia, fibrosis and diabetic retinopathy.

### BACKGROUND TO THE INVENTION

The phosphoinositide 3-kinase (PI3 Kinase) pathway plays a central role in regulating many biological events through phosphorylation of the plasma membrane lipid phosphatidylinositol 3,4-biphosphate (PtdIns(4,5)P₂), to produce the key second messenger phosphatidyl 3,4,5-triphosphate (PtdIns(3,4,5)P₃), [Krystal G., Semin. Immunol., 2000, 12, 397-403]. Such phosphorylation of PtdIns(4,5)P₂ at the D-3 position of the inositol ring in response to cell stimulation by growth factors and hormones sets in motion a coordinated set of events leading to cell proliferation, cell growth, cell cycle entry, cell migration, membrane trafficking, glucose transport, superoxide production and cell survival. PI3 Kinases can be classified into three sub-families according to structure and substrate specificity [Vanhaesebroeck et al., Annu. Rev. Biochem., 2001, 70, 535-602]. The best characterised of these sub-families are class I PI3 kinases consisting of two subgroups. Class IA and Class IB enzymes signal downstream of receptor tyrosine kinases and heterotrimeric G-protein-coupled receptors respectively. Class IA PI3 kinases consist of a p85 regulatory subunit and a p110 catalytic subunit [Cantley, Science, 2002, 296, 1655-1657]. There are three catalytic isoforms (p110α, p110β and p110δ) and five regulatory isoforms (p85α, p85β and p55γ, which are encoded by specific genes, and p55α and p50α that are produced by alternate splicing of the p85α gene), [Ward and Finan, Current Opinion in Pharmacology, 2003, 3, 426]. The regulatory subunit maintains the p110 catalytic subunit in a low-activity state in quiescent cells and mediates its activation by the interaction of the SH2 domain and phoshotyrosine residues of other proteins. In addition, p85 binds and integrates signals from intracellular proteins such as protein kinase C (PKC), SHP1, Rac, Rho and mutated Ras providing an integration point for activation of p110 and downstream molecules. The only Class IB PI3 Kinase identified to date is the p110γ catalytic subunit, complexed with a p101 regulatory protein. All class I PI3 kinases possess intrinsic protein kinase activity with p110 autophosphorylation and phosphorylation of p85 downregulating the activity of the complex.

Class II PI3 Kinases are monomeric proteins which lack regulatory subunits and utilise phosphatidylinositol (PtdIns) and phosphatidylinositol-4-monophosphate (PtdIns(4)P) as substrates, [Oudit et al, J. Mol. Cell. Cardiol. 2004, 37, 449]. Three mammalian class II isoforms have been identified PI3K-C2α, PI3K-C2β and PI3K-C2γ.

Class III PI3 kinases are heterodimeric species consisting of adaptor p150 and catalytic (Vps34, 100 KDa) subunits.

Signalling proteins with pleckstrin homology domains (PH) domains accumulate at sites of Class 1 PI3 Kinase activation by directly binding to PtdIns(3,4,5)P₃. PH domains are globular protein domains of about 100 amino acids and are found in a diverse array of proteins including kinases (Akt, PDK1, Btk), nucleotide exchange factors (e.g. Vav, GRP1, ARNO, Sos1), GTP-ase activating factors (e.g. GAP1^{m}, centaurins), phospholipases (e.g. PLCγ2). Of particular significance is the serine/threonine kinase Akt, one of the major direct downstream targets of Class I PI3 kinases. The PI3 kinase mediated production of PtdIns(3,4,5)P₃ in response to extracellular stimulation, leads to recruitment of Akt from the cytoplasm to the cell membrane through binding of it's PH domain to the membrane bound PtdIns(3,4,5)P₃. Such binding induces conformational changes in Akt facilitating phosphorylation at Thr 308 by PDK1 leading to it's activation. Akt modulates cell survival by both up regulating pro-survival pathways (e.g. CREB) and down regulating pro-apoptotic pathways (including BAD, procaspase-9 and Forkhead (FHKR) transcription factors). This PtdIns(3,4,5)P₃ signalling is negatively regulated by the lipid phosphatase PTEN (phosphatase and tensin homologue deleted on chromosome ten) which converts PtdIns(3,4,5)P₃ to PtdIns(4,5)P₂.

Mutations in the PI3 kinase pathway in cancer are common and have a role in neoplastic transformation. Amplification or mutation of the gene encoding p110a (PIK3CA) commonly occur in bowel cancer, ovarian cancer, head and neck and cervical squamous cancers, gastric and lung cancers, anaplastic oligodendrogliomas, glioblastoma multiforme and medulloblastomas. Somatic missense mutations of PIK3CA are frequent in HER2-amplified and hormone receptor positive breast cancers. Akt and PTEN are also targets of frequent genomic and epigenetic changes in human cancer. The PI3 kinase-Akt pathway is also required for the oncogenic effects of EGFR.

Leukocyte chemotaxis toward sites of inflammation is primarily mediated by cytokine signalling. It has been shown that regulation of p110γ is mediated via the p101 adapter, engaged by G_{iβγ} subunits released by activation of GPCRs [Stephens et al, Cell 1997, 89, 105-114]. Class IB PI3 kinase deficient mice, PI3Kγ^{-/-}, showed in vitro and in vivo impaired migration of neutrophils and macrophages towards chemoattractants [Hirsch et al, Science 2000, 287, 1049-1053 and Li et al., Science 2000, 287, 1046-1049]. p110γ deficient neutrophils are unable to produce PtdIns(3,4,5)P₃ when stimulated with GPCR agonists such as f MLP, C5a or IL-8. It has been reported [Weiss-Haljiti J. Biol. Chem. 2004, 279, 43273-43284] that in macrophages, the chemokine RANTES activates the small GTPase Rac and its target PAK2. This response depends on Gi activation and primarily on the subsequent activation of PI3 kinase γ and Rac. Rac constitutes a subfamily of the Rho family of monomeric GTPases and cycle between active GTP-bound (Rac^{GTP}) and inactive GDP-bound (Rac^{GDP}) states. Rho GTPases integrate signals from cellular receptors and membrane components to regulate the cytoskeleton dynamics required for cell locomotion during chemotaxis, phagocytosis and many other cellular responses. A loss of this PI3 kinase γ response could impair the ability of lymphocytes to make cellular contact with antigen presenting cells, thus impeding cell survival and the ability of cells to respond to immune stimulation [Costello et al, Nature Immunol 2002, 3, 1082].

The present invention relates to compounds which are inhibitors of PI3 Kinase. The compounds are thus of use in medicine, for example in the treatment and prophylaxis of neoplastic, immune and inflammatory disorders. The compounds are characterised by the presence in the molecule of an amino acid motif or an amino acid ester motif which is hydrolysable by an intracellular carboxylesterase. Compounds of the invention having the lipophilic amino acid ester motif cross the cell membrane, and are hydrolysed to the acid by the intracellular carboxylesterases. The polar hydrolysis product accumulates in the cell since it does not readily cross the cell membrane. Hence the PI3 kinase activity of the compound is prolonged and enhanced within the cell. The compounds of the invention are related to the PI3 kinase inhibitors encompassed by the disclosures in International Patent Application WO03072552 but differ therefrom in that the present compounds have the amino acid ester motif referred to above.

### DETAILED DESCRIPTION OF THE INVENTION

According to the invention there is provided a compound of formula (I):

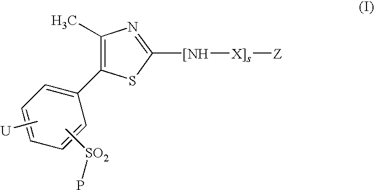

wherein:
s is 0 or 1;
U is hydrogen or halogen;
X is —(C═O), an optionally substituted divalent phenylene, pyridinylene, pyrimidinylene, or pyrazinylene radical, or a bond;
P is optionally substituted C₁-C₆ alkyl and Z is —(CH₂)_{z}—X₁-L₁-NHCHR₁R₂; or Z is optionally substituted C₁-C₆ alkyl and P is —(CH₂)ₙ—X₁-L₁-NHCHR₁R₂;
R₁ is a carboxylic acid group (—COOH), or an ester group which is hydrolysable by one or more intracellular carboxylesterase enzymes to a carboxylic acid group;
R₂ is the side chain of a natural or non-natural alpha amino acid;
X₁ is (i) a bond; —NR₄C(═O)NR₅— or —NR₄S(═O)₂—; or except when X is —(C═O)— (ii) —C(═O)—, —S(═O)₂—, or —S(═O)₂NR₄— wherein R₄ and R₅ are independently hydrogen or optionally substituted C₁-C₆ alkyl;
z is 0 or 1;
L₁ represents a divalent radical of formula -(Alk¹)ₘ(O)ₙ(Alk²)ₚ- wherein m, n and p are independently 0 or 1,
   Q is (i) an optionally substituted divalent mono- or bicyclic carbocyclic or heterocyclic radical having 5-13 ring members, or (ii), in the case where both m and p are 0, a divalent radical of formula —X²-Q¹- or -Q¹-X²- wherein X² is —O—, —S— or —NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
   Alk¹ and Alk² independently represent optionally substituted divalent C₃-C₇ cycloalkyl radicals, or optionally substituted straight or branched, C₁-C₆ alkylene, C₂-C₆ alkenylene , or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl.

Compounds of formula (I) above may be prepared in the form of salts, especially pharmaceutically acceptable salts, N-oxides, hydrates, and solvates thereof. Any claim to a compound herein, or reference herein to “compounds of the invention”, “compounds with which the invention is concerned”, “compounds of formula (I)”, and the like, includes salts, N-oxides, hydrates, and solvates of such compounds.

Although the above definitions potentially include molecules of high molecular weight, it is preferable, in line with general principles of medicinal chemistry practice, that the compounds with which this invention is concerned should have molecular weights of no more than 600.

In another broad aspect the invention provides the use of a compound of formula (I) as defined above, or an N-oxide, salt, hydrate or solvate thereof in the preparation of a composition for inhibiting the activity of a PI3 kinase, particularly PI3 kinase α, and PI3 kinase γ.

The compounds with which the invention is concerned may be used for the inhibition of PI3 kinase activity, particularly PI3 kinase α and PI3 kinase γ activity, ex vivo or in vivo.

In one aspect of the invention, the compounds of the invention may be used in the preparation of a composition for the treatment of neoplastic, immune and inflammatory disorders. For example the compounds may be used in treatment of cell-proliferation disease such as cancers, including bowel cancer, ovarian cancer, head and neck and cervical squamous cancers, gastric and lung cancers, anaplastic oligodendrogliomas, glioblastoma multiforme and medulloblastomas; in inflammatory and immune disease such as rheumatoid arthritis, psoriasis, inflammatory bowel disease, Crohn's disease, ulcerative colitis, chronic obstructive pulmonary disease, asthma, multiple sclerosis, diabetes, atopic dermatitis, graft versus host disease, systemic lupus erythematosus and others; in cardiovascular disorders such as myocardial ischemia, reperfusion injury and others. The foregoing disorders are known to be associated with PI3 Kinase activity.

In another aspect, the invention provides a method for the treatment of the foregoing disease types, which comprises administering to a subject suffering such disease an effective amount of a compound of the invention.

A particular subset of the compounds of the invention consists of those of formula (IA):

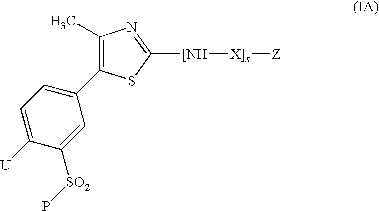

wherein U, P, X, Z and s are as defined in relation to formula (I).

### Terminology

The term “ester” or “esterified carboxyl group” means a group R^{X}O(C═O)— in which R^{X} is the group characterising the ester, notionally derived from the alcohol R^{X}OH.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to b carbon atoms, at least one double bond, and two unsatisfied valences.

As used herein the term “Cₐ-C_{b} alkynyl” wherein a and b are integers refers to straight chain or branched chain hydrocarbon groups having from a to b carbon atoms and having in addition one triple bond. For a=2 and b=6 this term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (Cₐ-C_{b})alkynylene radical” wherein a and b are integers refers to a divalent hydrocarbon chain having from a to b carbon atoms, and at least one triple bond.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

A “divalent phenylene, pyridinylene, pyrimidinylene, or pyrazinylene radical” is a benzene, pyridine, pyrimidine or pyrazine ring, with two unsatisfied valencies, and includes 1,3-phenylene, 1,4-phenylene, and the following:

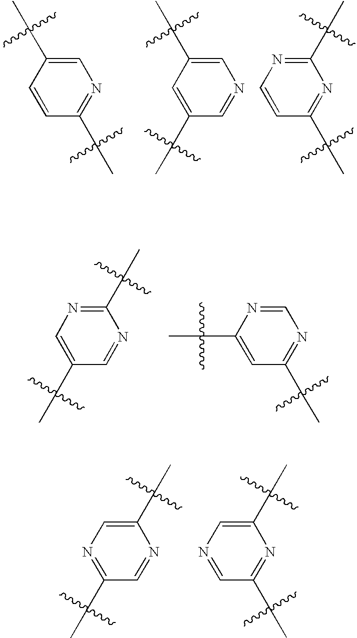

Unless otherwise specified in the context in which it occurs, the term “substituted” as applied to any moiety herein means substituted with up to four compatible substituents, each of which independently may be, for example, (C₁-C₆)alkyl, (C₂-C₆)alkenyl, (C₂-C₆)alkynyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo (═O), phenyl, phenoxy, monocyclic heteroaryl or heteroaryloxy with 5 or 6 ring atoms, —COOR^{A}, —COR^{A}, —OCOR^{A}, —SO₂R^{A}, —CONR^{A}R^{B}, —SO₂NR^{A}R^{B}, —NR^{A}R^{B}, OCONR^{A}R^{B}, NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl group or, in the case where R^{A} and R^{B} are linked to the same N atom, R^{A} and R^{B} taken together with that nitrogen may form a cyclic amino ring such as a morpholinyl, piperidinyl or piperazinyl ring. Where the substituent is phenyl, phenoxy or monocyclic heteroaryl or heteroaryloxy with 5 or 6 ring atoms, the phenyl or heteroaryl ring thereof may itself be substituted by any of the above substituents except phenyl phenoxy, heteroaryl or heteroaryloxy. An “optional substituent” or “substituent” may be one of the foregoing specified groups.

The term “side chain of a natural or non-natural alpha-amino acid” refers to the group R^{Y} in a natural or non-natural amino acid of formula NH₂—CH(R^{Y})—COOH.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, α-amino-n-butyric acid, 3,4-dihydroxyphenylalanine, homoserine, α-methylserine, ornithine, pipecolic acid, and thyroxine.

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When R₂ in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

Examples of side chains of non-natural alpha amino acids include those referred to below in the discussion of suitable R₂ groups for use in compounds of the present invention.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like. For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds of the invention which contain one or more actual or potential chiral centres, because of the presence of asymmetric carbon atoms, can exist as enantiomers or as a number of diastereoisomers with R or S stereochemistry at each chiral centre. The invention includes all such enantiomers and diastereoisomers and mixtures thereof.

The esters of the invention are converted by intracellular esterases to the carboxylic acid. Both the esters and carboxylic acids may have PI3 kinase inhibitory activity in their own right. The compounds of the invention therefore include not only the ester, but also the corresponding carboxylic acid hydrolysis products.

### The Substituents in Formulae (I) and (IA)

One of P and Z is optionally substituted C₁-C₆ alkyl, while the other is a radical —(CH₂)_{z}—X₁-L₁-NHCHR₁R₂. In one particular case, P is optionally substituted C₁-C₆ alkyl while Z is a radical —(CH₂)_{z}—X₁-L₁-NHCHR₁R₂. Optionally substituted C₁-C₆ alkyl radicals P and Z include optionally substituted methyl, ethyl, and n- and iso-propyl. For example P may be methyl.

U may be, for example, hydrogen, fluoro, chloro or bromo. Presently chloro is preferred.

X may be, for example —(C═O), a bond, 1,3-phenylene, 1,4-phenylene, or one of the following divalent radicals:

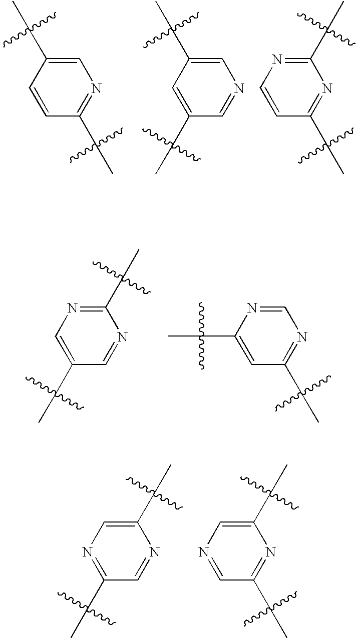

In the P or Z radical —(CH₂)_{z}—X₁-L₁-NHCHR₁R₂, the —NHCHR₁R₂ part is of course an alpha amino acid or ester motif, linked via its amino group to L₁ of the linker part —(CH₂)_{z}—X₁-L₁-. That linker part —(CH₂)_{z}—X₁-L₁- arises as a result of the particular chemistry used to attach the alpha amino acid or ester motif to the rest of the molecule.

R₁ may be a carboxylic acid group. Although compounds of this class may be administered as the carboxylic acid or a salt thereof, it is preferred that they be generated in the cell by the action of an intracellular esterase on a corresponding compound in which R₁ is an ester group.

The ester group R₁ must be one which in the compound of the invention is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes other enzymes such as biphenylhydrolase (BPH) may also have a role in hydrolysing the conjugates. In general, if the carboxylesterase hydrolyses the free amino acid ester to the parent acid it will, also hydrolyse the ester motif when covalently linked to the rest of the molecule. Hence, the broken cell assay described herein provides a straightforward, quick and simple first screen for esters which have the required hydrolysis profile. Ester motifs selected in that way may then be re-assayed in the same carboxylesterase assay when incorporated in the PI3 inhibitor of the invention via the chosen conjugation chemistry, to confirm that it is still a carboxylesterase substrate in that background.

Subject to the requirement that they be hydrolysable by intracellular carboxylesterase enzymes, examples of particular ester groups R₁ include those of formula —(C—O)OR₇ wherein R₇ is R₈R₉R₁₀C— wherein
(i) R₈ is hydrogen or optionally substituted (C₁-C₃)alkyl-(Z¹)ₐ-[(C₁-C₃)alkyl]_{b}- or (C₂-C₃)alkenyl-(Z¹)ₐ-[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen.

Within these classes, R₁₀ is often hydrogen. Specific examples of R₇ include methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl or methoxyethyl. Currently preferred is where R₇ is cyclopentyl.

Macrophages are known to play a key role in inflammatory disorders through the release of cytokines in particular TNFα and IL-1 (van Roon et a/Arthritis and Rheumatism, 2003, 1229-1238). In rheumatoid arthritis they are major contributors to the maintenance of joint inflammation and joint destruction. Macrophages are also involved in tumour growth and development (Naldini and Carraro Curr Drug Targets Inflamm Allergy, 2005, 3-8). Hence agents that selectively target macrophage cell proliferation could be of value in the treatment of cancer and autoimmune disease. Targeting specific cell types would be expected to lead to reduced side-effects. The inventors have discovered a method of targeting PI3 inhibitors to macrophages which is based on the observation that the way in which the esterase motif is linked to the inhibitor determines whether it is hydrolysed, and hence whether or not it accumulates in different cell types. Specifically it has been found that macrophages contain the human carboxylesterase hCE-1 whereas other cell types do not. In the compounds of the invention when the nitrogen of the esterase motif —NHCHR₁R₂ is not directly linked to a carbonyl (—C(═O)—), the ester will only be hydrolysed by hCE-1 and hence the inhibitors will only accumulate in macrophages. Herein, unless “monocyte” or “monocytes” is specified, the term macrophage or macrophages will be used to denote macrophages (including tumour associated macrophages) and/or monocytes.

Subject to the requirement that the ester group R₁ be hydrolysable by intracellular carboxylesterase enzymes, the identity of the side chain group R₂ is not critical.

Examples of amino acid side chains include

C₁-C₆ alkyl, phenyl, 2,- 3-, or 4-hydroxyphenyl, 2,- 3-, or 4-methoxyphenyl, 2,- 3-, or 4-pyridylmethyl, benzyl, phenylethyl, 2-, 3-, or 4-hydroxybenzyl, 2,- 3-, or 4-benzyloxybenzyl, 2,-3-, or 4- C₁-C₆ alkoxybenzyl, and benzyloxy(C₁-C₆alkyl)-groups;
the characterising group of a natural a amino acid, in which any functional group may be protected;
groups -[Alk]ₙR₁₄ where Alk is a (C₁-C₆)alkyl or (C₂-C₆)alkenyl group optionally interrupted by one or more —O—, or —S— atoms or —N(R₁₅)— groups [where R₁₅ is a hydrogen atom or a (C₁-C₆)alkyl group], n is 0 or 1, and R₁₄ is an optionally substituted cycloalkyl or cycloalkenyl group;
a benzyl group substituted in the phenyl ring by a group of formula —OCH₂COR₁₆ where R₁₆ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino, the residue of an amino acid or acid halide, ester or amide derivative thereof, said residue being linked via an amide bond, said amino acid being selected from glycine, α or β alanine, valine, leucine, isoleucine, phenylalanine, tyrosine, tryptophan, serine, threonine, cysteine, methionine, asparagine, glutamine, lysine, histidine, arginine, glutamic acid, and aspartic acid;
a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; and
a group —CRₐR_{b}R_{c} in which:
each of Rₐ, R_{b} and Rₑ is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆) alkyl, —S(C₂-C₆)alkenyl, —SO(C₂-C₆)alkenyl, —SO₂(C₂-C₆)alkenyl or a group -Q²-W wherein Q² represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

Examples of particular R₂ groups include hydrogen (the glycine “side chain”), benzyl, phenyl, cyclohexylmethyl, cyclohexyl, pyridin-3-ylmethyl, tert-butoxymethyl, iso-butyl, sec-butyl, tert-butyl, 1-benzylthio-1-methylethyl, 1-methylthio-1-methylethyl, 1-mercapto-1-methylethyl, and phenylethyl. Presently preferred R₂ groups include phenyl, benzyl, cyclohexyl and iso-butyl.

For compounds of the invention which are to be administered systemically, esters with a slow rate of carboxylesterase cleavage are preferred, since they are less susceptible to pre-systemic metabolism. Their ability to reach their target tissue intact is therefore increased, and the ester can be converted inside the cells of the target tissue into the acid product. However, for local administration, where the ester is either directly applied to the target tissue or directed there by, for example, inhalation, it will often be desirable that the ester has a rapid rate of esterase cleavage, to minimise systemic exposure and consequent unwanted side effects. In the compounds of this invention, if the carbon adjacent to the alpha carbon of the alpha amino acid ester ester is monosubstituted, i.e. R₂ is —CH₂R^{z} (R^{z} being the mono-substituent) then the esters tend to be cleaved more rapidly than if that carbon is di- or tri-substituted, as in the case where R₂ is, for example, phenyl or cyclohexyl.

As mentioned, in the P or Z radical —(CH₂)_{z}—X₁-L₁-NHCHR₁R₂, linker part —(CH₂)_{z}—X₁-L₁- arises from the particular chemistry strategy chosen to link the amino acid ester motif —NHCHR₁R₂ aminothiazole part of the molecule. Clearly the chemistry strategy for that coupling may vary widely, and thus many combinations of the variables L₁, X₁ and z are possible. The precise combination of variables making up the linking chemistry between the amino acid ester motif and aminothiazole part will often be irrelevant to the primary binding mode of the compound as a whole. On the other hand, that linkage chemistry will in some cases pick up additional binding interactions with the enzyme.

It may also be mentioned that the structure of the linker part —(CH₂)Z-X₁-L₁- may vary depending on the identity of the X part of the compounds of the invention. For example when X is a carbonyl radical —(C═O)— it is unlikely for reasons of compatibility that z will be 0 when X₁ is —C(═O)—, —S(═O)₂—, —C(═O)NR₄—, or —S(═O)₂NR₄—.

It should also be noted that the benefits of the amino acid ester motif described above (facile entry into the cell, esterase hydrolysis within the cell, and accumulation within the cell of active carboxylic acid hydrolysis product) are best achieved when the linkage between the amino acid ester motif and the aminothizole part is not a substrate for peptidase activity within the cell, which might result in cleavage of the amino acid from the molecule. Of course, stability to intracellular peptidases is easily tested by incubating the compound with disrupted cell contents, and analysing for any such cleavage.

With the foregoing general observations in mind, taking the variables making up the radical —(CH₂)_{z}—X¹-L₁- in turn:
z may be 0 or 1, so that a methylene radical linked to the aminothiazolyl part is optional;
In the radical L₁, examples of Alk¹ and Alk² radicals, when present, include —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—C≡C—, —C≡CCH₂—, CH₂C≡C—, and CH₂C≡CCH₂. Additional examples of Alk¹ and Alk² include —CH₂W—, —CH₂CH₂W— —CH₂CH₂WCH₂—, —CH₂CH₂WCH(CH₃)—, —CH₂WCH₂CH₂—, —CH₂WCH₂CH₂WCH₂—, and —WCH₂CH₂— where W is —O—, —S—, —NH—, —N(CH₃)—, or —CH₂CH₂N(CH₂CH₂OH)CH₂—. Further examples of Alk¹ and Alk² include divalent cyclopropyl, cyclopentyl and cyclohexyl radicals.

In L₁, when n is 0, the radical is a hydrocarbon chain (optionally substituted and perhaps having an ether, thioether or amino linkage). Presently it is preferred that there be no optional substituents in L₁. When both m and p are 0, L₁ is a divalent mono- or bicyclic carbocyclic or heterocyclic radical with 5-13 ring atoms (optionally substituted). When n is 1 and at least one of m and p is 1, L₁ is a divalent radical including a hydrocarbon chain or chains and a mono- or bicyclic carbocyclic or heterocyclic radical with 5-13 ring atoms (optionally substituted). When present, Q may be, for example, a divalent phenyl, naphthyl, cyclopropyl, cyclopentyl, or cyclohexyl radical, or a mono-, or bi-cyclic heterocyclic radical having 5 to 13 ring members, such as piperidinyl, piperazinyl, indolyl, pyridyl, thienyl, or pyrrolyl radical, but 1,4-phenylene is presently preferred.

Specifically, in some embodiments of the invention, L¹, m and p may be 0 with n being 1. In other embodiments, n and p may be 0 with m being 1. In further embodiments, m, n and p may be all 0. In still further embodiments m may be 0, n may be 1 with Q being a monocyclic heterocyclic radical, and p may be 0 or 1. Alk¹ and Alk², when present, may be selected from —CH₂—, —CH₂CH₂—, —CH₂CH₂CH₂— and —CH₂CH₂CH₂ CH₂— and Q, when present may be 1,4-phenylene.

Specific examples of the radical -L₁-X₁— [CH₂]_{z}- include —C(═O)— and —C(═O)NH— as well as —(CH₂)ᵥ—, —(CH₂)₁₋₀—, —C(═O)—(CH₂)ₙ—, —C(═O)—(CH₂)ᵥO—, —C(═O)—NH—(CH₂)_{w}—, —C(═O)—NH—(CH₂)_{w}O—

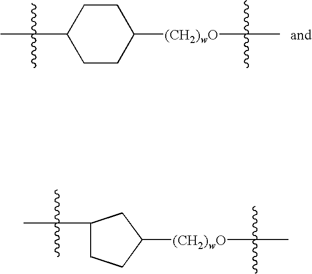

wherein v is 1, 2, 3 or 4 and w is 1, 2 or 3, such that -L¹-X¹—[CH₂]_{z}—, is —CH₂—, —CH₂CH₂—, —CH₂CH₂CH₂—, —CH₂CH₂CH₂CH₂—, —CH₂O—, —CH₂CH₂O—, —CH₂CH₂CH₂O—, —CH₂CH₂CH₂CH₂O—, —C(═O)—CH₂—, —C(═O)—CH₂O—, —C(═O)—NH—CH₂—, or —C(═O)—NH—CH₂O—.

A specific subset of compounds of the invention consists of those of formula (IB), particularly those of formula (IC):

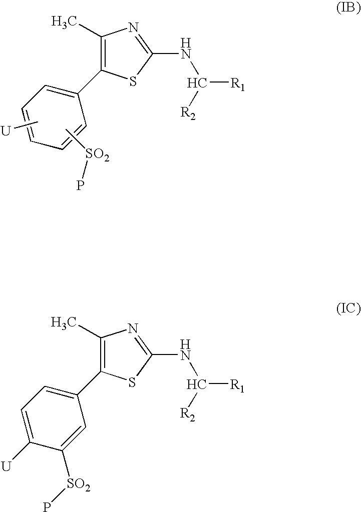

wherein U, P, R₁ and R₂ are as defined, discussed or specifically mentioned above.

A further specific subset of compounds of the invention consists of those of formula (ID), particularly those of formula (IE):

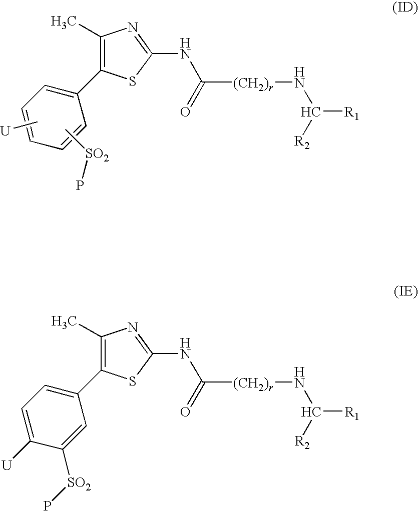

wherein U, P, R₁ and R₂ are as defined, discussed or specifically mentioned above and r is 1, 2, 3 or 4. In compounds (ID) and (1E), U may be, for example, chloro, and P may be, for example methyl.

Yet another specific subset of compounds of the invention consists of those of formula (IF):

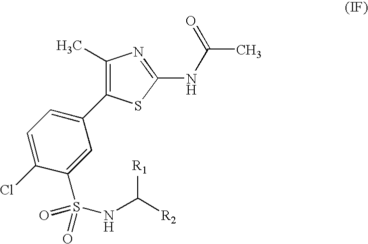

wherein R₁ and R₂ are as defined in relation to formula (I) above or as more particularly discussed above.

As mentioned above, the compounds with which the invention is concerned are inhibitors of the PI3 kinase family, particularly PI3 kinase 4 and/or PI3 kinase y, and are therefore of use in the treatment of neoplastic, immune and inflammatory disease in humans and other mammals.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavorings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agent can be dissolved in the vehicle.

### Synthesis

The compounds of the invention may be prepared by a number of processes described in the Examples hereinafter. In the reactions described below, it may be necessary to protect reactive functional groups, for example hydroxyl, amino and carboxyl groups, where these are desired in the final product, to avoid their unwanted participation in the reactions [see for example Greene, T. W., “Protecting Groups in Organic Synthesis”, John Wiley and Sons, 1999]. Conventional protecting groups may be used in conjunction with standard practice.

One general route to some of the compounds of the invention is typified by Scheme 1:

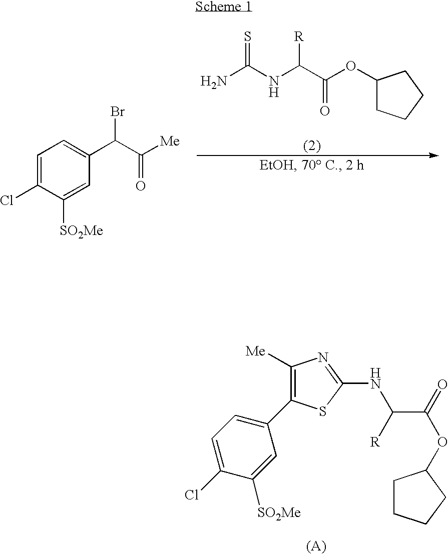

Thus, thioureas of formula (2) and analogous reagents may be condensed with 1-bromo-1-(4-chloro-3-methanesulfonylphenyl)-propan-2-one and analogous reagents using methods described in WO03072552 to give the thiazole (A) and analogous compounds. The thioureas of formula (2) may be prepared by the reaction of amino acid esters analogous to formula (3) with thiophosgene in the presence of a mineral base, such as potassium or calcium carbonate, in an inert chlorinated solvent such as dichloromethane as shown in Scheme 2. Such procedures are set forth in texts familiar to those skilled in the art [e.g. March's Advanced Organic Chemistry, John Wiley and Sons, 1992].

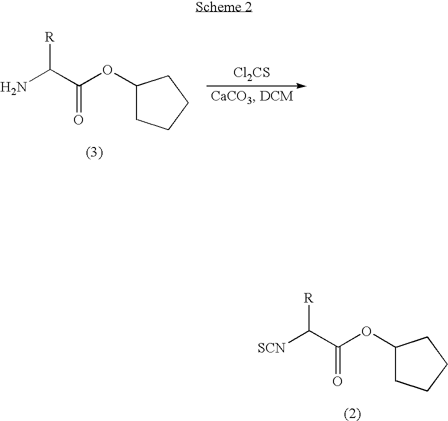

Compounds of general formula (B) and analogous compounds of the invention may be prepared by the route summarised in Scheme 3. Thus treatment of the carbamates of formula (6) with trifluoroacetic acid in dichloromethane at ambient temperature yields the thiazoles (B). The carbamates (6) may be prepared by the coupling of an appropriately substituted carboxylic acid of general formula (5) with the aminothiazole of formula (4) [WO03072552], using a carbodiimide such as EDC and HOBt in an aprotic solvent such as DMF. It will be seen by those skilled in the art that several methods of amide bond formation may be applicable to this process. [March's Advanced Organic Chemistry [John Wiley and Sons, 1992].

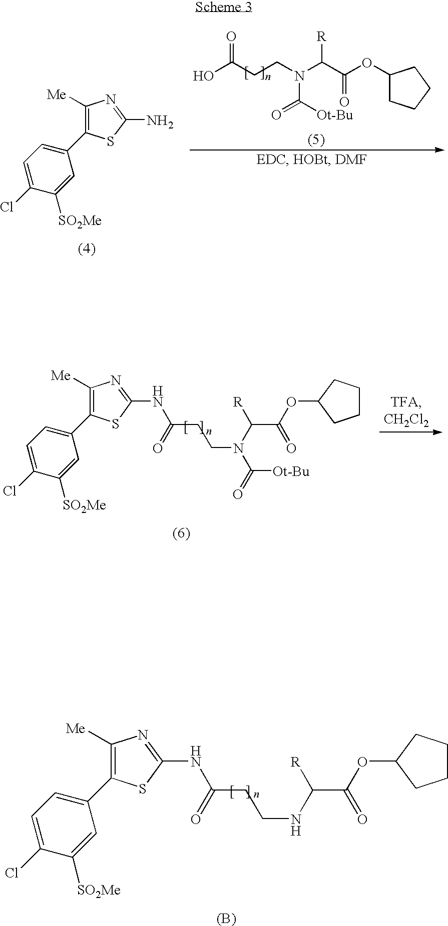

Carboxylic acids of general formula (5) may be prepared as described in Scheme 4. Thus, hydrogenation of benzyl esters of formula (7) over a palladium catalyst in a solvent such as THF or ethanol at ambient temperature yields acids of formula (5). Benzyl esters of formula (7) may be prepared by the reaction of amino acid esters of formula (8) with di-tert-butoxycarbonate in an inert solvent such as dichloromethane or THF, in the presence of an amine base such as triethylamine. The amino acid esters (8) may be prepared by the alkylation of a primary amino acid ester, such as L-leucine cyclopentyl ester, with a bromoalkanoic acid benzyl ester (9) in the presence of sodium iodide and potassium carbonate in an aprotic solvent such as DMF.

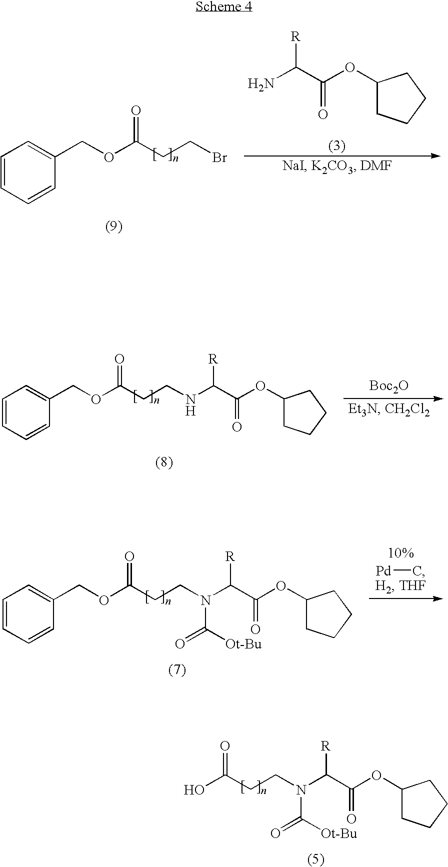

Amino acids of general formula (C) and analogous compounds of the invention may be prepared by treatment of esters of general formula (A) by hydrolysis utilizing a mineral hydroxide such as aqueous lithium or sodium hydroxide in the presence of an organic co-solvent such as tetrahydrofuran, as shown in Scheme 5. Similarly esters of formula (B) as described in Scheme 3 may be N-protected as, for example, the t-butoxycarbonyl derivatives, then also be hydrolysed to the N-protected acids, which in turn may then be deprotected for example by treatment with trifluoroacetic acid in dichloromethane at ambient temperature to give amino acids of general formula (D), as shown in Scheme 6.

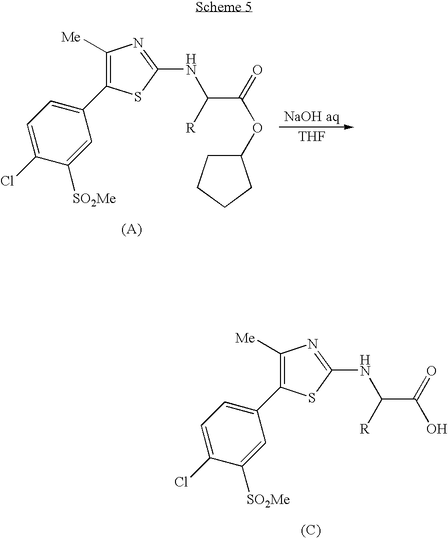

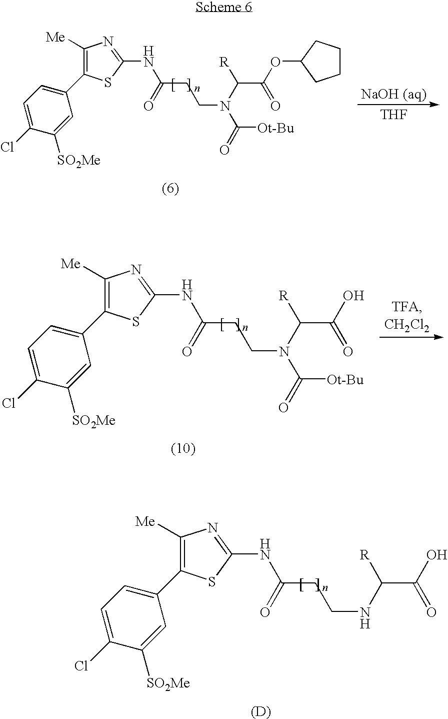

In a further route, amino acid esters of general formula (E) and analogous esters may be prepared by methods described in Scheme 7. Thioureas of formula (11) may be condensed with 1-bromo-1-(4-chloro-3-methanesulfonylphenyl)-propan-2-one using methods described in WO03072552 to give the thiazole of formula (12). Deprotection of the aldol protection functionality of (12) under aqueous acidic conditions may be employed to yield the aldehyde of formula (13). Aldehyde (13) may the be reacted with an amino acid ester to give the compound of general formula (E) and analogous esters

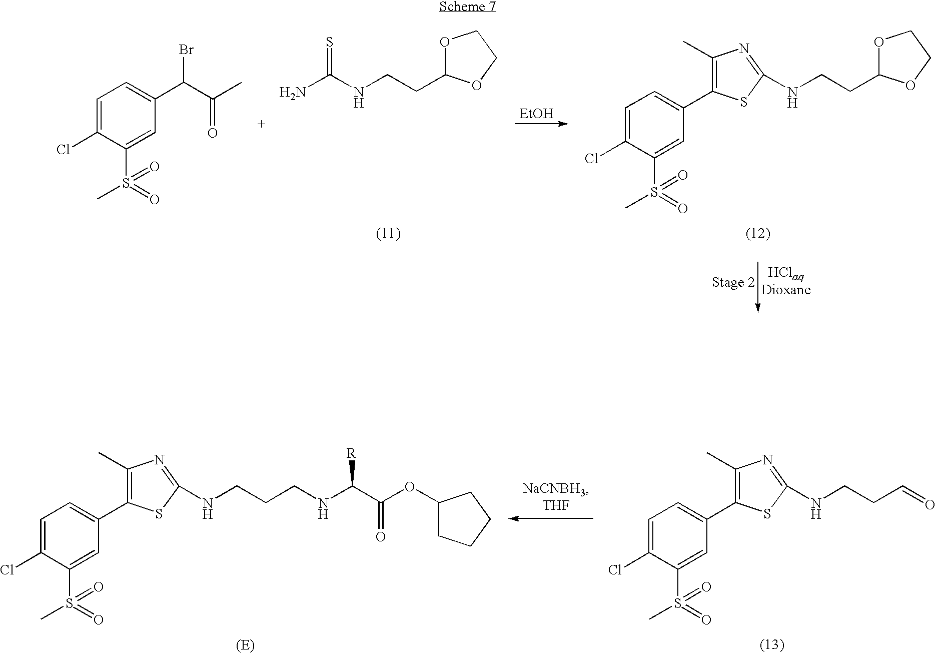

In a further route, amino acid esters of formula (F) and analogous esters may be prepared by methods described in Scheme 8. Thus sulphonyl chloride (14) may be reacted with an amino acid ester of general formula (15) to give a sulphonamide of formula (16). Treatment of (16) with bromine in dioxane can be employed to give the bromoketone of general formula (17). Reaction of (17) with acetylthiourea in ethanol may be employed to give esters of general formula (F) and analogous esters.

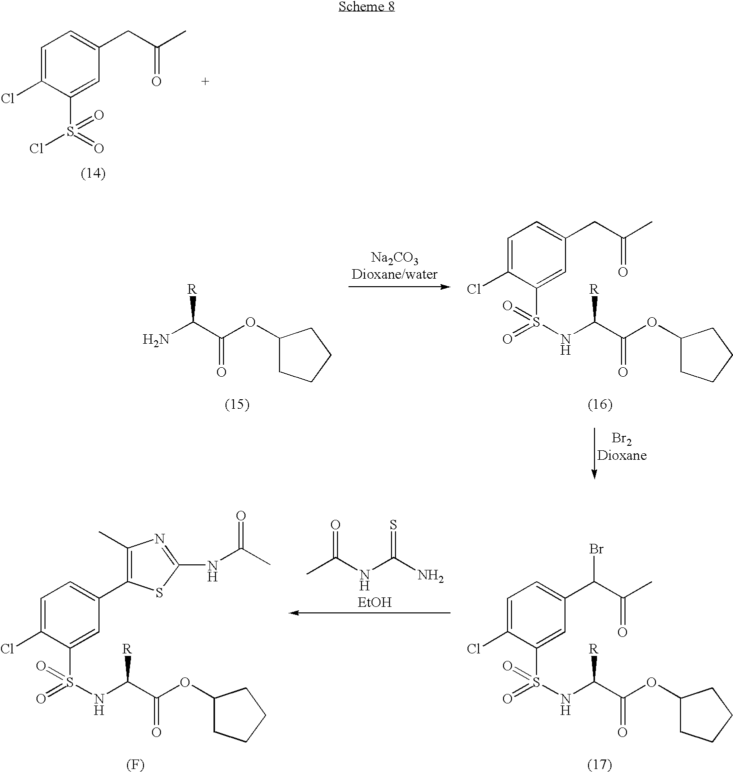

The following examples illustrate the preparation and properties of some specific compounds of the invention. All temperatures are in ° C. The following abbreviations are used:

MeOH=methanol
EtOH=ethanol
EtOAc=ethyl acetate
Boc=tert-butoxycarbonyl
DCM=dichloromethane
DMF=dimethylformamide
DMSO=dimethyl sulfoxide
TFA=trifluoroacetic acid
THF=tetrahydrofuran
Na₂CO₃=sodium carbonate
HCl=hydrochloric acid
DIPEA=diisopropylethylamine
NaH=sodium hydride
NaOH=sodium hydroxide
NaHCO₃=sodium hydrogen carbonate
Pd/C=palladium on carbon
TME=tert-butyl methyl ether
N₂=nitrogen
PyBop=benzotriazole-1-yl-oxy-tris-pyrrolidino-phosphonium hexafluorophosphate
Na₂SO₄=sodium sulphate
Et₃N=triethylamine
NH₃=ammonia
TMSCl=trimethylchlorosilane
NH₄Cl=ammonium chloride
LiAlH₄=lithium aluminium hydride
pyBrOP=bromo-tris-pyrrolidino phosphoniumhexafluorophosphate
MgSO₄=magnesium sulfate
ⁿBuLi=n-butyllithium
CO₂=carbon dioxide
EDCl=N-(3-Dimethylaminopropyl)-N′-ethylcarbodiimide hydrochloride
Et₂O=diethyl ether
LiOH=lithium hydroxide
HOBT=1-hydroxybenzotriazole

### ELS=Evaporative Light Scattering

TLC=thin layer chromatography
ml=milliliter(s)
g=gram(s)
mg=milligram(s)
mol=mole(s)
mmol=millimole(s)
LCMS=high performance liquid chromatography/mass spectrometry
NMR=nuclear magnetic resonance
RT=room temperature

Microwave irradiation was carried out using a CEM Discover focused microwave reactor. Solvents were removed using a GeneVac Series I without heating or a Genevac Series II with VacRamp at 30° C. or a Buchi rotary evaporator. Purification of compounds by flash chromatography column was performed using silica gel, particle size 40-63 μm (230-400 mesh) obtained from Silicycle. Purification of compounds by preparative HPLC was performed on Gilson systems using reverse phase ThermoHypersil-Keystone Hyperprep HS C18 columns (12 μm, 100×21.2 mm), gradient 20-100% B (A=water/0.1% TFA, B=acetonitrile/0.1% TFA) over 9.5 min, flow=30 ml/min, injection solvent 2:1 DMSO:acetonitrile (1.6 ml), UV detection at 215 nm.

¹H NMR spectra were recorded on a Bruker 400 MHz AV or a Bruker 300 MHz AV spectrometer in deuterated solvents. Chemical shifts δ are in parts per million. Thin-layer chromatography (TLC) analysis was performed with Kieselgel 60 F₂₅₄ (Merck) plates and visualized using UV light.

Analytical HPLCMS was performed on Agilent HP1100, Waters 600 or Waters 1525 LC systems using reverse phase Hypersil BDS C18 columns (5 μm, 2.1×50 mm), gradient 0-95% B (A=water/0.1% TFA, B=acetonitrile/0.1% TFA) over 2.10 min, flow=1.0 ml/min. UV spectra were recorded at 215 nm using a Gilson G1315A Diode Array Detector, G1214A single wavelength UV detector, Waters 2487 dual wavelength UV detector, Waters 2488 dual wavelength UV detector, or Waters 2996 diode array UV detector. Mass spectra were obtained over the range m/z 150 to 850 at a sampling rate of 2 scans per second or 1 scan per 1.2 seconds using Micromass LCT with Z-spray interface or Micromass LCT with Z-spray or MUX interface. Data were integrated and reported using OpenLynx and OpenLynx Browser software.

### Intermediates

The following amino acid esters were used as intermediates for the preparation of the examples described herein:

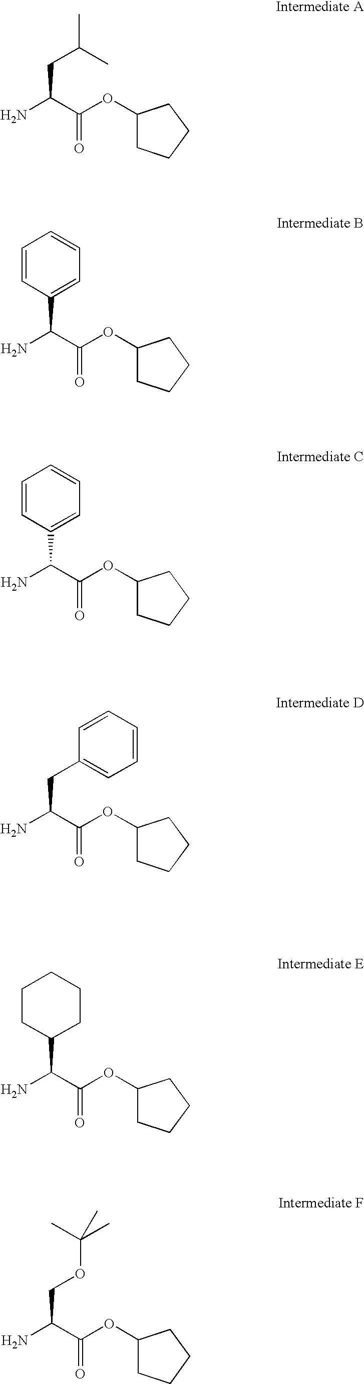

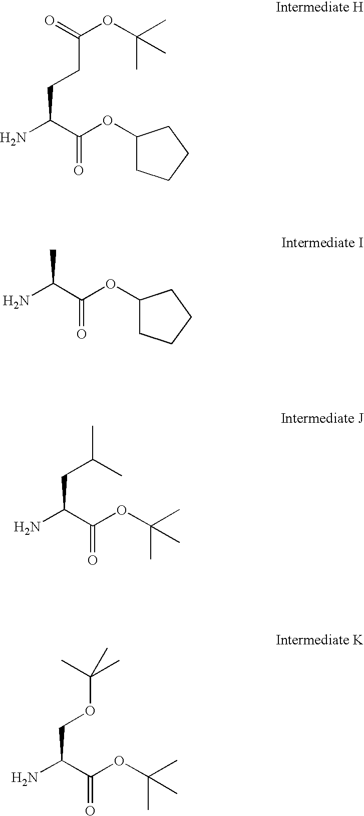

### Method for the Preparation of the Intermediates Above

### Method I. Used for the Preparation of Intermediates A, D, E and I

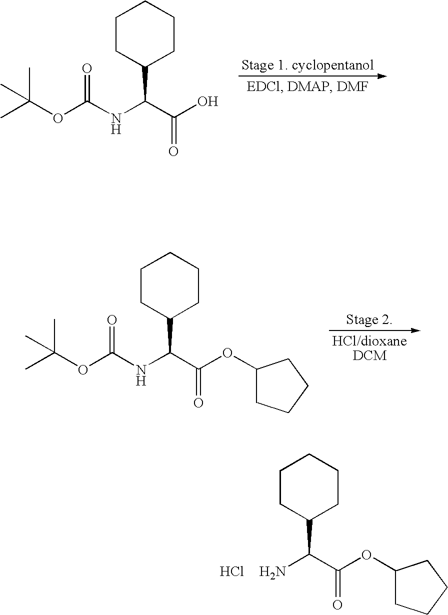

### Method II. Used for the Preparation of Intermediates B and C

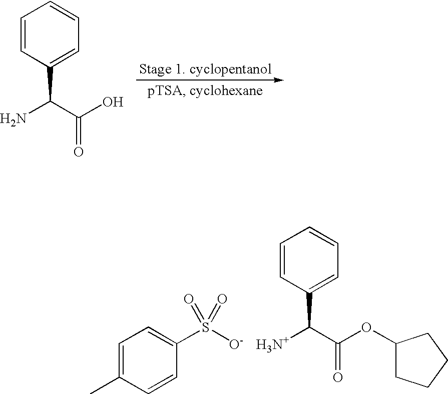

### Method III. Used for the Preparation of Intermediates F and H

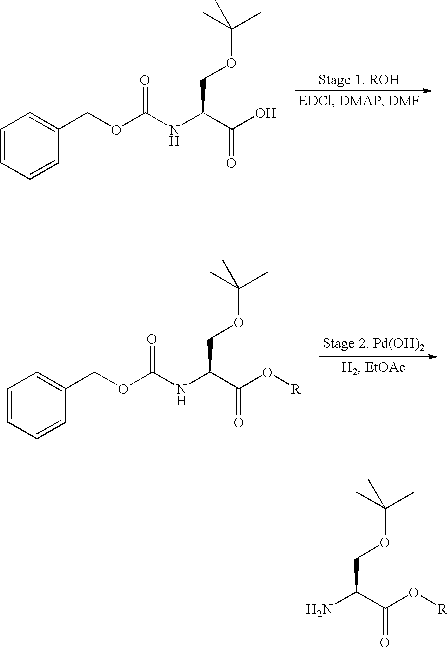

### Method I

### Exemplified for intermediate E, cyclopentyl (2S)-amino(cyclohexyl)acetate

### Stage

To a solution of (S)-2-tert-butoxycarbonylamino-3-cyclohexyl-propionic acid (5 g, 19.4 mmol) in DMF (50 ml) at 0° C. was added cyclopentanol (8.8 ml, 97.15 mmol), EDC (4.09 g, 21.37 mmol) and finally DMAP (237 mg, 1.94 mmol). The reaction mixture was warmed to RT and stirred for 18 h. The DMF was removed in vacuo to give a clear oil. This was separated between water and EtOAc. The organic phase was dried (MgSO₄) and concentrated in vacuo. The crude extract was purified by column chromatography (25% EtOAC in heptane) to yield the desired product as a clear oil (14.87 g, 55%). ¹H NMR (300 MHz, d6-DMSO) δ: 7.09 (1H, d), 5.08 (1H, t), 3.76 (1H, t), 1.50-1.85 (10H, br m), 1.39 (9H, s), 1.00-1.25 (9H, br m).

### Stage 2 (Intermediate E)

Stage 1 product (14.87 g, 45.69 mmol) was dissolved in DCM (100 ml) and treated with 4M HCl/dioxane (22.8 ml, 91.38 mmol) and the reaction mixture was stirred at RT for 24 h. The crude mixture was concentrated under reduced pressure to give an orange oil. This was triturated with Et₂O to give a white precipitate. This was further washed with Et₂O to give the desired product as a white powder (7.78 g, 65%). ¹H NMR (300 MHz, d₆-DMSO) δ: 8.45 (3H, br s), 5.22 (1H, t), 3.28 (1H, d), 1.95-1.50 (10H, br m), 1.30-0.90 (9H, br m).

Intermediates A, D, G and I were also prepared via this route. Data for each intermediate is given:

### Intermediate A Cyclopentyl L-leucinate m/z 200 [M+H]⁺, ¹H NMR (300 MHz, CDCl₃) δ: 0.90 (6H, t, J=6.4 Hz), 1.23-1.94 (11H, m), 3.38 (1H, dd, J=8.4, 5.9 Hz), 5.11-5.22 (1H, m).

### Intermediate D Cyclopentyl L-phenylalaninate

m/z 234 [M+H]⁺, ¹H NMR (300 MHz, CDCl₃) δ: 7.23-7.18 (5H, m), 5.44 (1H, m), 5.14 (1H, m), 3.44-3.34 (2H, m), 1.94-1.41 (8H, br m).

### Intermediate I Cyclopentyl L-alaninate

m/z 158 [M+H]⁺, ¹H NMR (300 MHz, CD₃OD) δ: 1.48 (3H, d), 1.70-1.77 (6H, m), 1.87-1.97 (2H, m), 4.03 (2H, q), 5.28 (1H, m).

### Method II

### Exemplified for Intermediate B, cyclopentyl (2S)-amino(phenyl)acetate tosylate

To a slurry of (S)-phenylglycine (5 g, 33.1 mmol) in cyclohexane (150 ml) was added cyclopentanol (29.84 ml, 331 mmol) and p-toluene sulfonic acid (6.92 g, 36.4 mmol). The reaction was fitted with a Dean-Stark receiver and heated to 135° C. for complete dissolution. After 12 h, the reaction was cooled to RT leading to the precipitation of a white solid. The solid was filtered and washed with EtOAc before drying under reduced pressure to give the required product as a white powder (11.01 g, 85%). ¹H NMR (300 MHz, d₆-DMSO) δ: 8.82 (2H, br s), 8.73 (1H, br s), 7.47 (7H, m), 7.11 (2H, d), 5.25 (1H, br s), 5.18 (1H, m), 2.29 (3H, s), 1.87-1.36 (8H, m).

Intermediate C was also prepared via this route. Data for this intermediate is given:

### Intermediate C Cyclopentyl (2R)-amino(phenyl)acetate tosylate salt

¹H NMR (300 MHz, d₆-DMSO) δ: 8.80 (2H, br s), 8.74 (1H, br s), 7.44 (7H, m), 7.13 (2H, d), 5.28 (1H, br s), 5.21 (1H, m), 2.26 (3H, s), 1.85-1.30 (8H, m).

### Method III

### Exemplified for Intermediate F, Cyclopentyl O-Tert-Butyl-L-Serinate

### Stage 1

To a solution of (S)-2-benzyloxycarbonylamino-3-tert-butoxy-propionic acid (25 g, 84.65 mmol) in DMF (250 ml) at 0° C. was added cyclopentanol (15.36 ml, 169.3 mmol), EDCl (17.85 g, 93.11 mmol) and finally DMAP (1.03 g, 8.46 mmol). The reaction mixture was warmed to RT and stirred for 18 h. The DMF was removed in vacuo to give a yellow oil. This was partitioned between water and EtOAc. The organic phase was dried (MgSO₄) and concentrated in vacuo. The crude extract was purified by column chromatography (25% EtOAC in heptane) to yield the desired product as a clear oil. This was used directly in the next stage without characterization.

### Stage 2

Stage 1 product was dissolved in EtOAc (150 ml), treated with Pd(OH)₂ (10 mol %) and stirred under an atmosphere of hydrogen for 32 h. Upon completion, the catalyst was removed by filtration through celite and the filtrate concentrated in vacuo to yield the desired product as a clear oil (15.96 g, 82% over two steps). ¹H NMR (300 MHz, d₆-DMSO) δ: 5.17 (1H, t), 3.45 (1H, m), 3.34 (2H, q), 1.90-1.50 (9H, br m), 1.08 (9H, s).

Intermediate H was also prepared via this route. Data for this intermediate is given:

### Intermediate H 5-tert-Butyl 1-cyclopentyl L-glutamate

¹H NMR (300 MHz, CDCl₃) δ: 1.47 (9H, s), 1.53-1.99 (10H, m), 2.38 (2H, t), 3.41 (1H, dd), 5.21 (1H, m).

Intermediates J and K are commercially available.

All the above intermediates were used in aminoacid coupling reactions as free bases. To an individual skilled in the art, it will be apparent that each free base can be prepared prepared by titration of the salts described above with a suitable inorganic base (e.g. NaHCO₃).

The following other intermediates were also used for the preparation of the examples described herein:

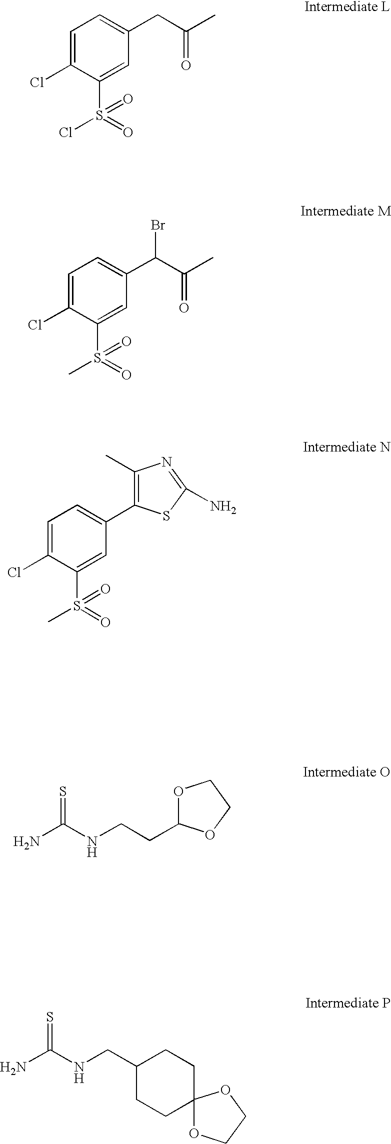

### Synthesis of Intermediates L, M and N

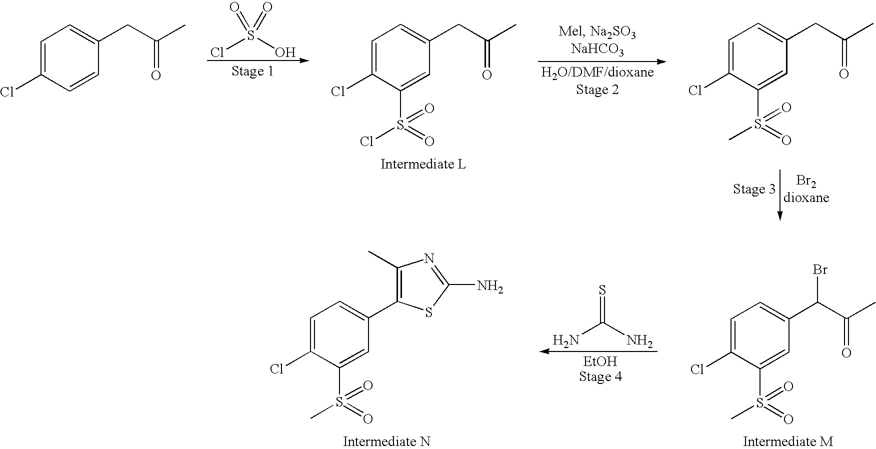

### Stage 1 Intermediate L

2-Chlorophenylacetone (4 g, 23.7 mmol) was added dropwise to chlorosulfonic acid (30 ml) at −10° C. The reaction was allowed to warm to RT and stirred for 36 h. Upon completion, the reaction was quenched by slow addition to crushed ice (500 ml). The aqueous solution was then extracted with EtOAc (3×100 ml) and the combined organics were dried (MgSO₄) and concentrated in vacuo to afford the title compound (6.7 g, 98%). m/z 289 [M+Na]⁺, ¹H NMR (300 MHz, CDCl₃) δ: 7.96 (1H, d, J=2.1 Hz), 7.62 (1H, d, J=8.7 Hz), 7.49 (1H, dd, J=2.1, 8.7 Hz), 3.86 (2H, s), 2.30 (3H, s).

### Stage 2

A mixture of Na₂SO₃ (5.97 g, 47.7 mmol) and NaHCO₃ (3.98 g, 47.4 mmol) in water (100 ml) was stirred at 70° C. To this was added a solution of stage 1 product (6.33 g, 23.7 mmol) in dioxane (200 ml). Stirring was then continued at 70° C. for 1 h. The reaction was then cooled to RT and concentrated in vacuo. The residue was re-dissolved in DMF (200 ml) and treated with MeI (2.95 ml, 47.7 mmol). The mixture was then heated to 40° C. for 1 h. Most of the solvent was then concentrated in vacuo and the residue poured into water (200 ml) and extracted with EtOAc (3×200 ml). The organics were washed with brine (200 ml), dried (MgSO₄) and concentrated to afford an orange oil (5.95 g, quant.). This was used in the next stage without further purification or characterization.

### Stage 3 Intermediate M 1-bromo-1-[4-chloro-3-(methylsulfonyl)phenyl]acetone

Bromine (912 μl, 17.8 mmol) was added dropwise to a solution of stage 2 product (5.95 g, 23.7 mmol) in 1,4-dioxane (150 ml) at RT. The reaction was stirred at RT for 24 h and the solvent was then removed in vacuo (maintaining the bath temp below 30° C.). The residue was dissolved in EtOAc (250 ml) and washed with sat NaHCO_{3(aq)} (200 ml), water (200 ml) and brine (200 ml) then dried (MgSO₄) and concentrated in vacuo to give an orange oil. This was purified by flash chromatography (EtOAc/heptane, 1:1) to afford the product as a clear oil (1.31 g, 17%). ¹H NMR (300 MHz, CDCl₃) δ: 2.46 (3H, s), 3.31 (3H, s), 5.43 (1H, s), 7.61 (1H, d, J=9.8 Hz), 7.43 (1H, dd, J=1.1, 9.8 Hz), 8.17 (1H, d, J=1.1 Hz).

### Stage 4 Intermediate N 5-[4-chloro-3-(methylsulfonyl)phenyl]-4-methyl-1,3-thiazol-2-amine

Thiourea (804 mg, 4 mmol) was added to a solution of stage 3 product (1.31 g, 4 mmol) in ethanol (40 ml) and the reaction stirred at 70° C. for 1.5 h. Upon cooling a white precipitate formed so it was isolated by filtration, washed with EtOH (10 ml) and Et₂O (10 ml) to afford the title compound as a cream-coloured powder (984 mg, 81%). ¹H NMR (300 MHz, CD₃OD) δ: 2.35 (3H, s), 3.74 (3H, s), 7.60-7.83 (2H, m), 8.14 (1H, d, J=1.1 Hz).

### Synthesis of Intermediates O and P (Exemplified for Intermediate O):

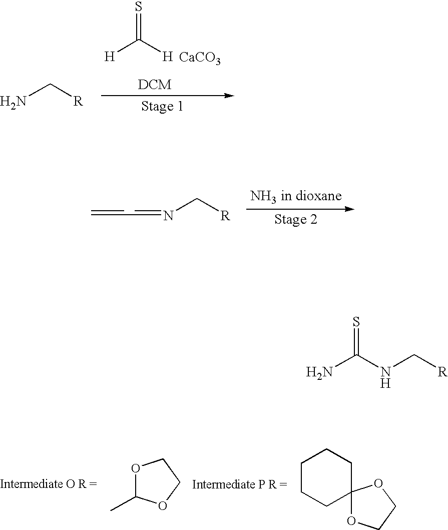

### Stage 1

CaCO₃ (1.36 g, 13.66 mmol) was added to a vigorously stirred solution of 2-[1,3]dioxolan-2-yl-ethylamine (1.0 g, 8.54 mmol) in DCM (20 ml) and water (10 ml). Thiophosgene (0.85 ml, 11.10 mmol) was added dropwise over 5 min and the resultant biphasic mixture was vigorously stirred at RT for 18 h. The reaction mixture was diluted with water (40 ml) and the organic phase was separated. The aqueous layer was extracted with DCM (2×40 ml) and the combined organic layers were washed with water (50 ml) followed by brine (50 ml), dried (Na₂SO₄) and concentrated in vacuo to give a yellow oil (1.36 g, 100%). This material was used in the next step without further purification.

### Stage 2 Intermediate O 1-[2-(1,3-dioxolan-2-yl)ethyl]thiourea

Stage 1 product (1.36 g, 8.58 mmol) was dissolved in 0.5M NH₃ in dioxane solution (51.5 ml) and was stirred at RT for 36 h. The reaction mixture was evaporated to dryness under reduced pressure and was purified by flash chromatography (2% to 10% MeOH in DCM) to give the product as a yellow oil (1.55 g, 100%). LCMS purity 100%, m/z 177 [M+H]⁺.

Intermediate P was also prepared via this route from 1-(1,4-dioxaspiro[4.5]dec-8-yl)methanamine. Data for this intermediate is given:
1-(1,4-Dioxaspiro[4.5]dec-8-ylmethyl)thiourea (Intermediate P)

LCMS purity 100%, m/z 215 [M+H]⁺

### EXAMPLES

The following are representative examples of the compounds claimed by the invention.

### Example 1

### Cyclopentyl N-{5-[4-chloro-3-(methylsulfonyl)phenyl]-4-methyl-1,3-thiazol-2-yl}-L-phenylalaninate

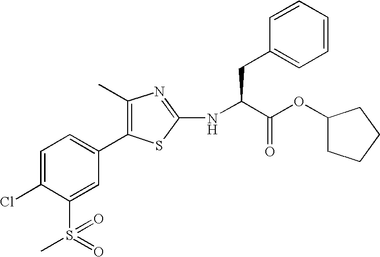

The compound of Example 1 was prepared by the following methodology:

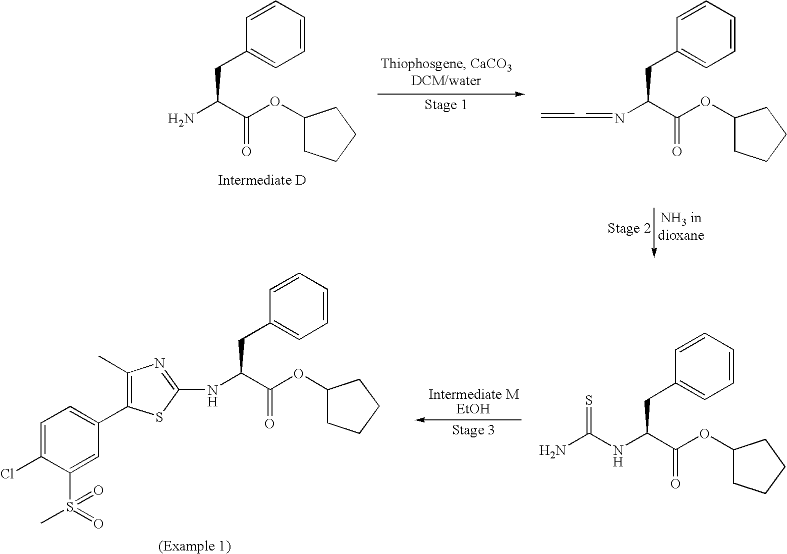

### Stage 1

To a solution of Intermediate D (2.0 g, 8.58 mmol) in a mixture of DCM (20 ml) and water (10 ml) was added CaCO₃ (1.37 g, 13.73 mmol). The resulting white suspension was stirred and thiophosgene (0.85 ml, 11.16 mmol) was added slowly (over 5 mins) and the reaction was stirred at RT for 1 h. The reaction mixture was then diluted with water (20 ml). The organic layer was isolated and the aqueous layer was extracted with DCM (20 ml). The combined organic layers were washed with brine (20 ml), dried (Na₂SO₄) and evaporated in vacuo to give an orange coloured oil (2.1 g, 89%). This was used in the next stage without further purification or characterisation.

### Stage 2

The product from Stage 1 (0.3 g, 1.09 mmol) was dissolved in 0.5M NH₃ in dioxane (6.55 ml) and was stirred at RT for 2 h. The reaction mixture was evaporated to dryness under reduced pressure and was purified by flash chromatography (2% MeOH in DCM) to afford the product as a yellow oil (0.2 g, 62%). LCMS purity 92%, m/z 293 [M+H]⁺.

### Stage 3

A mixture of Intermediate M (50 mg, 0.15 mmol) and the product of Stage 2 (49 mg, 0.17 mmol) in EtOH (2 ml) was stirred at 70° C. for 1 h. The reaction mixture was evaporated to dryness, partitioned between EtOAc (5 ml) and sat NaHCO_{3(aq)} (1 ml). The EtOAc layer was dried (Na₂SO₄) and concentrated in vacuo to give a dark orange oil. Purification by flash chromatography (30% EtOAc in heptane) gave the desired product as a yellow solid (51 mg, 64%). LCMS purity 95%, m/z 519 [M+H]⁺, ¹H NMR (400 MHz, CD₃OD) δ: 1.40-1.75 (8H, m), 2.15 (3H, s), 2.90-3.10 (2H, m) 3.35 (3H, s), 4.05-4.55 (1H, m), 5.00-5.05 (1H, m), 7.05-7.20 (5H, m), 7.50-7.55 (2H, m), 7.90-7.95 (1H, m).

### Example 2

### Cyclopentyl (2S)-({5-[4-chloro-3-(methylsulfonyl)phenyl]-4-methyl-1,3-thiazol-2-yl}amino)(phenyl)acetate

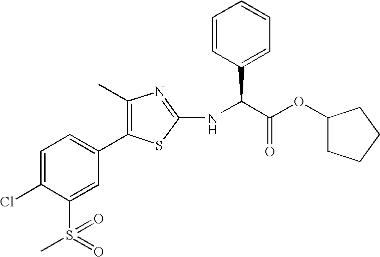

Example 2 was prepared from Intermediate B and Intermediate M using a similar methodology as described for the compound of Example 1. LCMS purity 98%, m/z 505 [M+H]⁺, ¹H NMR (400 MHz, CD₃OD) δ: 1.45-1.90 (8H, m), 2.30 (3H, s), 3.35 (3H, s), 5.15-5.25 (1H, m), 5.45-5.50 (1H, m), 7.35-7.50 (5H, m), 7.65-7.70 (2H, m), 8.05-8.10 (1H, m).

### Example 3

### Cyclopentyl N-{5-[4-chloro-3-(methylsulfonyl)phenyl]-4-methyl-1,3-thiazol-2-yl}-L-leucinate

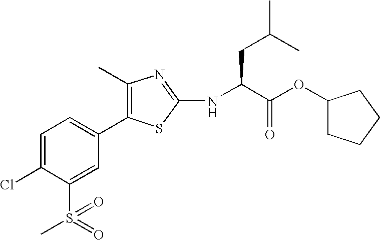

Example 3 was prepared from Intermediate A and Intermediate M using a similar methodology as described for the compound of Example 1. LCMS purity 98%, m/z 485 [M+H]⁺, ¹H NMR (400 MHz, CD₃OD) δ: 0.85-0.95 (6H, m), 1.45-1.80 (11H, m), 2.15 (3H, s), 3.35 (3H, s), 4.25-4.35 (1H, m), 5.05-5.15 (1H, m), 7.55-7.60 (2H, m), 7.80-7.90 (1H, m).

### Example 4

### Cyclopentyl O-tert-butyl-N-({5-[4-chloro-3-(methylsulfonyl)phenyl]-4-methyl-1,3-thiazol-2-yl}carbamoyl)-L-serinate

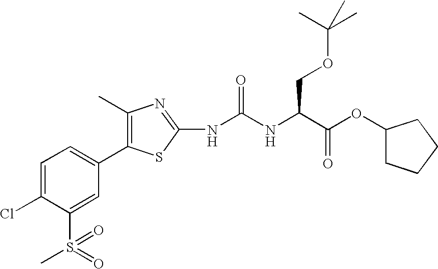

Example 4 was prepared by the following methodology

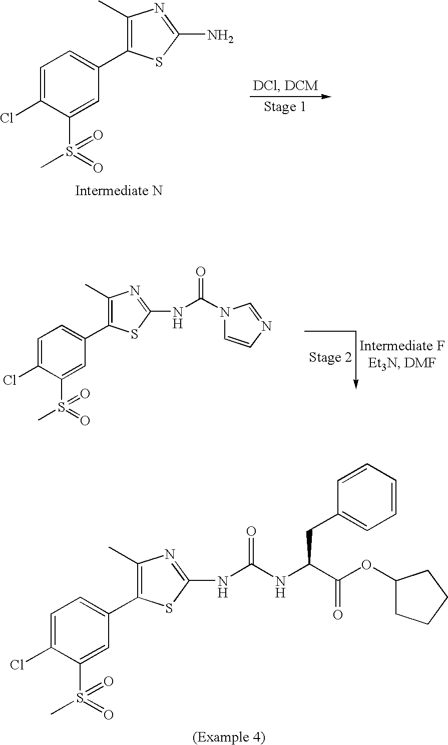

### Stage 1

A suspension of Intermediate N (203 mg, 0.67 mmol) and DCl (160 mg, 1 mmol) in DCM (8 ml) was heated to 40° C. under N₂ atmosphere for 3 h. A precipitate had formed and was filtered and washed with DCM (10 ml) to afford a white solid (240 mg, 90%). ¹H NMR (300 MHz, CD₃OD) δ: 8.16 (1H, s), 7.72-7.79 (3H, m), 7.09 (2H, s), 3.36 (3H, s), 2.39 (3H, s).

### Stage 2

To a suspension of stage 1 product (80 mg, 0.202 mmol) and Intermediate F (46 mg, 0.202 mmol) in DMF (3 ml) was added Et₃N (57 μl, 0.404 mmol). The reaction was stirred at RT for 1 h after which time the solvent was removed in vacuo and the resulting residue purified by prep HPLC (MeCN/water) to afford the title compound (6 mg, 5%). LCMS purity 95%, m/z 558/560 [M+H]⁺, ¹H NMR (300 MHz, CD₃OD) δ: 8.15 (1H, d, J=1.9 Hz), 7.75-7.73 (2H, m), 5.27-5.21 (1H, m), 4.50 (1H, t, J=2.9 Hz), 3.91 (1H, dd, J=8.9, 2.8 Hz), 3.66 (1H, dd, J=8.9, 3.1 Hz), 3.36 (3H, s), 2.39 (3H, s), 1.92-1.59 (8H, m), 1.22 (9H, s).

### Example 5

### Cyclopentyl N-({5-[4-chloro-3-(methylsulfonyl)phenyl]-4-methyl-1,3-thiazol-2-yl}carbamoyl)-L-phenylalaninate

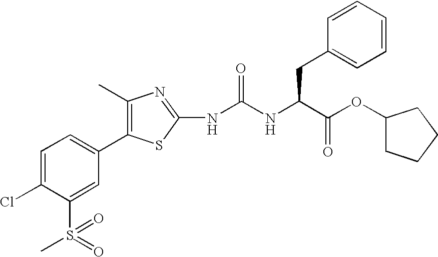

Example 5 was prepared from Intermediate N and Intermediate D using a similar methodology as described for Example 4. LCMS purity 100%, m/z 562/564 [M+H]⁺, ¹H NMR (400 MHz, CD₃OD) δ: 1.55-1.95 (8H, m), 2.35 (3H, s), 3.10-3.20 (2H, m), 3.35 (3H, s), 4.60-4.65 (1H, m), 5.15-5.25 (1H, m), 7.20-7.35 (5H, m), 7.70-7.80 (2H, m), 8.15-8.20 (1H, m).

### Example 6

### Cyclopentyl N-[4-({5-[4-chloro-3-(methylsulfonyl)phenyl]-4-methyl-1,3-thiazol-2-yl}amino)-4-oxobutyl]-L-leucinate

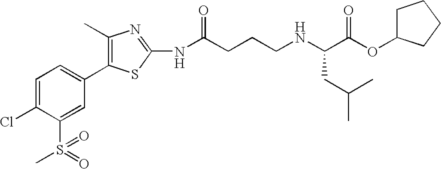

Example 6 was prepared by the following methodology

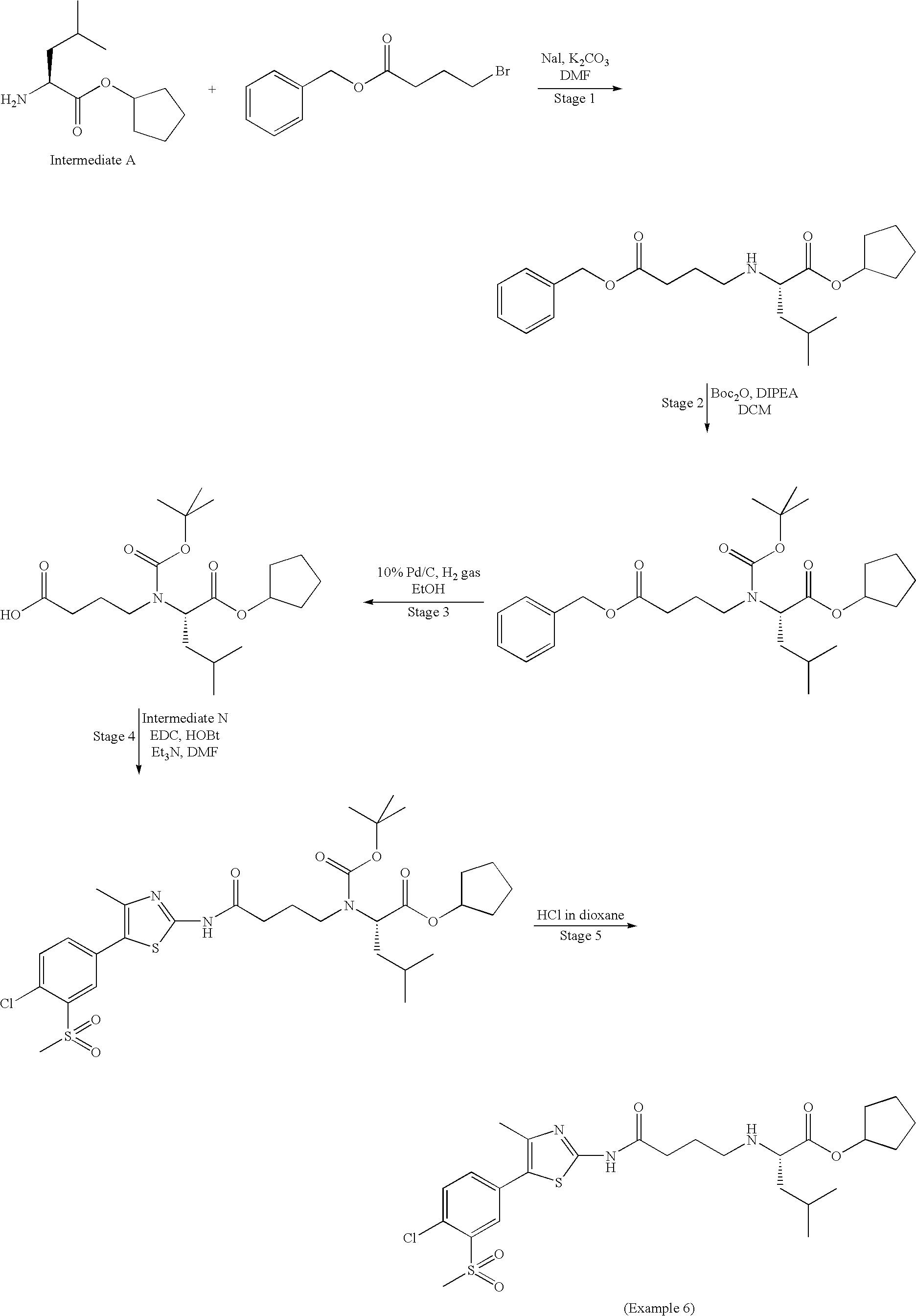

### Stage 1

To a stirred suspension of Intermediate A (1.2 g, 3.24 mmol) in DMF (10 ml) was added 4-bromo-butyric acid benzyl ester (1 g, 3.89 mmol), NaI (0.486 g, 3.24 mmol) and K₂CO₃ (0.89 g, 6.48 mmol). Stirring at RT was continued for 1.5 h. The reaction mixture was diluted with EtOAc (100 ml) and washed with water (2×50 ml). The EtOAc layer was then dried (Na₂SO₄), filtered and concentrated in vacuo. Purification by flash chromatography (0.06% NH₃/2.94% MeOH/97% DCM) afforded the desired product as a yellow oil (917 mg, 75%). m/z 376 [M+H]⁺.

### Stage 2

A mixture of stage 1 product (100 mg, 0.266 mmol), Boc₂O (69.7 mg, 0.32 mmol), and DIPEA (0.051 ml, 0.293 mmol) in DCM (2 ml) was stirred at RT for 18 h. The reaction mixture was washed with 0.5M HCl_{aq} (2 ml) followed by sat NaHCO_{3(aq)} (1 ml), dried (Na₂SO₄), filtered and concentrated in vacuo. Purification by preparative TLC (7.5% EtOAc/heptane) afforded the desired product (100 mg, 79%). m/z 476 [M+H]⁺.

### Stage 3

A mixture of stage 2 product (100 mg, 0.21 mmol), and 10% Pd/C (10% w/w) in EtOH (15 ml) was stirred under H₂ (balloon) at RT for 4 h. The reaction mixture was filtered through a pad of celite, washed with EtOH (20 ml) and concentrated in vacuo to give a white solid. To remove residual EtOH the solid was dissolved in toluene/THF mixture (5/1) (20 ml) and re-concentrated in vacuo to afford the desired product as a white powder (64 mg, 79%). m/z 386 [M+H]⁺, ¹H NMR (400 MHz, CDCl₃) δ: 0.90 (6H, s), 1.35 (9H, s), 2.35 (2H, m), 2.90-3.50 (2H, m), 3.95 (1H, m), 5.10 (1H, m).

### Stage 4

To a stirred mixture of stage 3 product (65 mg, 0.168 mmol), EDC (48 mg, 0.25 mmol) and HOBt (27 mg, 0.20 mmol) in DMF (0.5 ml) was added a solution of Intermediate N (51 mg, 0.168 mmol) in DMF (0.5 ml) at RT. Et₃N (0.035 ml, 0.25 mmol) was added and stirring was continued for 18 h. The reaction mixture was diluted with water (10 ml) and extracted with EtOAc (15 ml). The EtOAc layer was washed with water (2×5 ml), dried (Na₂SO₄), filtered and concentrated in vacuo. Purification by preparative TLC (65% EtOAc/heptane) afforded the desired product (25 mg, 22%). m/z 670/672 [M+H]⁺.

### Stage 5

A solution of stage 4 product (10 mg, 0.0149 mmol) in 20% TFA in DCM (0.3 ml) was allowed to stand at RT for 3 h. After completion the reaction mixture was concentrated in vacuo to afford the title compound (10 mg, 100%). LCMS purity 96%, m/z 570/572 [M+H]⁺, ¹H NMR (400 MHz, CD₃OD) δ: 0.90-1.05 (6H, m), 1.50-1-90 (11H, m), 1.95-2.05 (2H, m), 2.30 (3H, s), 2.60 (2H, m), 2.95-3.15 (2H, m), 3.35 (3H, s), 3.85-4.00 (1H, m), 5.20-5.30 (1H, m), 7.60-7.80 (2H, m), 8.05 (1H, s).

### Example 7

### Cyclopentyl N-[4-({5-[4-chloro-3-(methylsulfonyl)phenyl]-4-methyl-1,3-thiazol-2-yl}amino)-4-oxobutyl]-L-alaninate

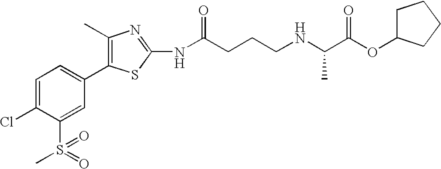

Example 7 was prepared from Intermediate N and Intermediate I using a similar methodology as described for the compound of Example 6. LCMS purity 95%, m/z 529 [M+H]⁺, ¹H NMR (300 MHz, CD₃OD) δ: 8.15 (1H, d, J=1.7 Hz), 7.80-7.72 (2H, m), 5.36-5.29 (1H, m), 4.16-4.06 (1H, m), 3.35 (3H, s), 3.22-3.14 (2H, m), 2.73 (2H, t, J=6.8 Hz), 2.41 (3H, m), 2.17-2.05 (2H, m), 1.99-1.62 (9H, m), 1.57 (3H, d, J=7.2 Hz).

### Example 8

### (4S)-4-{[4-({5-[4-Chloro-3-(methylsulfonyl)phenyl]-4-methyl-1,3-thiazol-2-yl}amino)-4-oxobutyl]amino}-5-(cyclopentyloxy)-5-oxopentanoic acid

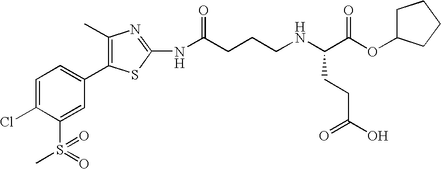

Example 8 was prepared from Intermediate N and Intermediate H using a similar methodology as described for the compound of Example 6. LCMS purity 93%, m/z 586 [M+H]⁺, ¹H NMR (300 MHz, CD₃OD) δ: 8.06 (1H, d, J=1.5 Hz), 7.69-7.66 (2H, m), 5.27-5.20 (1H, m), 4.03 (1H, dd, J=8.4, 5.0 Hz), 3.26 (3H, s), 3.17-3.05 (2H, m), 2.67 (2H, t, J=6.7 Hz), 2.54-2.37 (2H, m), 2.33 (3H, s), 2.28-2.13 (1H, m), 2.12-1.95 (3H, m), 1.92-1.78 (2H, m), 1.77-1.48 (6H, m).

### Example 9

### Cyclopentyl (2R)-{[4-({5-[4-chloro-3-(methylsulfonyl)phenyl]-4-methyl-1,3-thiazol-2-yl}amino)-4-oxobutyl]amino}(phenyl)acetate

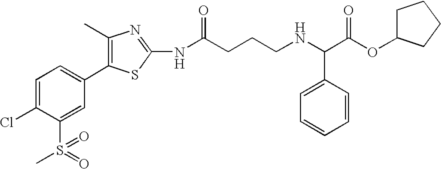

Example 9 was prepared from Intermediate N and Intermediate C using a similar methodology as described for the compound of Example 6. LCMS purity 94%, m/z 590/592 [M+H]⁺, ¹H NMR (400 MHz, CD₃OD) δ: 1.35-2.20 (10H, m), 2.35 (3H, s), 2.65-2.75 (2H, m), 2.95-3.20 (2H, m), 3.35 (3H, s), 5.15-5.20 (1H, m), 5.30-5.35 (1H, m), 7.45-7.55 (5H, m), 7.75-7.80 (2H, m), 8.15 (1H, s).

### Example 10

### Cyclopentyl (2S)-{[4-({5-[4-chloro-3-(methylsulfonyl)phenyl]-4-methyl-1,3-thiazol-2-yl}amino)-4-oxobutyl]amino}(phenyl)acetate

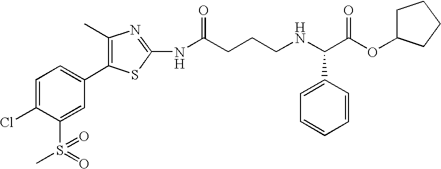

Example 10 was prepared from Intermediate N and Intermediate B using a similar methodology as described for the compound of Example 6. LCMS purity 99%, m/z 590/592 [M+H]⁺, ¹H NMR (400 MHz, CD₃OD) δ: 1.35-2.20 (10H, m), 2.35 (3H, s), 2.65-2.75 (2H, m), 2.95-3.20 (2H, m), 3.35 (3H, s), 5.15-5.25 (1H, m), 5.30-5.40 (1H, m), 7.45-7.55 (5H, m), 7.75-7.80 (2H, m), 8.15 (1H, s).

### Example 11

### Cyclopentyl N-[4-({5-[4-chloro-3-(methylsulfonyl)phenyl]-4-methyl-1,3-thiazol-2-yl}amino)-4-oxobutyl]-L-phenylalaninate

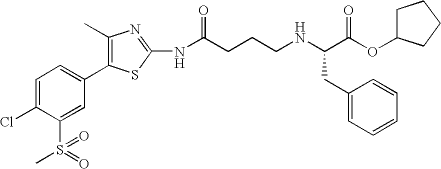

Example 11 was prepared from Intermediate N and Intermediate D using a similar methodology as described for the compound of Example 6. LCMS purity 92%, m/z 604/606 [M+H]⁺, ¹H NMR (400 MHz, CD₃OD) δ: 1.20-1.80 (8H, m), 1.95-2.10 (2H, m), 2.35 (3H, s), 2.60 (2H, m), 2.95-3.25 (4H, m), 3.40 (3H, s), 4.15-4.30 (1H, m), 5.05-5.15 (1H, m), 7.15-8.10 (8H, m).

### Example 12

### Cyclopentyl O-tert-butyl-N-[4-({5-[4-chloro-3-(methylsulfonyl)phenyl]-4-methyl-1,3-thiazol-2-yl}amino)-4-oxobutyl]-L-serinate

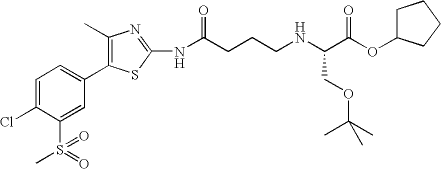

Example 12 was prepared from Intermediate N and Intermediate F using a similar methodology as described for the compound of Example 6. LCMS purity 98%, m/z 600 [M+H]⁺, ¹H NMR (300 MHz, CD₃OD) δ: 8.09 (1H, d, J=1.3 Hz), 7.52-7.48 (2H, m), 5.26-5.18 (1H, m), 4.05 (1H, q, J=7.2 Hz), 4.00-3.95 (1H, m), 3.92-3.85 (1H, m), 3.77 (1H, dd, J=9.8, 3.0 Hz), 3.24 (3H, s), 3.21-3.11 (2H, m), 2.84-2.65 (2H, m), 2.33 (3H, s), 2.21-2.07 (2H, m), 1.88-1.75 (2H, m), 1.73-1.48 (6H, m), 1.09 (9H, s).

### Example 13

### Cyclopentyl (2S)-{[4-({5-[4-chloro-3-(methylsulfonyl)phenyl]-4-methyl-1,3-thiazol-2-yl}amino)-4-oxobutyl]amino}(cyclohexyl)acetate

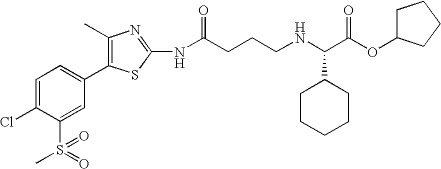

Example 13 was prepared from Intermediate N and Intermediate E using a similar methodology as described for the compound of Example 6. LCMS purity 100%, m/z 596/598 [M+H]⁺, ¹H NMR (300 MHz, CD₃OD) δ: 8.15 (1H, d, J=1.3 Hz), 7.76-7.73 (2H, m), 5.32-5.26 (1H, m), 3.54-3.46 (2H, m), 3.37 (3H, s), 3.02-2.83 (2H, m), 2.66 (2H, t, J=6.7 Hz), 2.41 (3H, s), 2.09-1.99 (2H, m), 1.97-1.87 (2H, m), 1.83-1.63 (10H, m), 1.36-1.23 (6H, m).

### Example 14

### tert-Butyl N-[4-({5-[4-chloro-3-(methylsulfonyl)phenyl]-4-methyl-1,3-thiazol-2-yl}amino)-4-oxobutyl]-L-leucinate

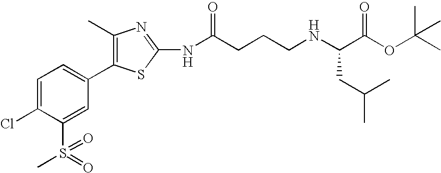

Example 14 was prepared from Intermediate N and Intermediate J using a similar methodology as described for the compound of Example 6. The final stage Boc deprotection was performed using 2M HCl in Et₂O. LCMS purity 92%, m/z 558/560 [M+H]⁺, ¹H NMR (400 MHz, CD₃OD) δ: 0.75-0.90 (6H, m), 1.30-1.45 (11H, m), 1.55-1.65 (1H, m), 1.75-1.85 (2H, m), 2.30 (3H, s), 2.40-2.65 (4H, m), 3.00-3.10 (1H, m), 3.35 (3H, s), 7.60-7.70 (2H, m), 8.05 (1H, s).

### Example 15

### tert-Butyl O-tert-butyl-N-[4-({5-[4-chloro-3-(methylsulfonyl)phenyl]-4-methyl-1,3-thiazol-2-yl}amino)-4-oxobutyl]-L-serinate

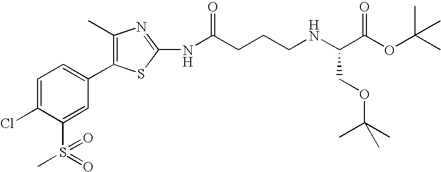

Example 15 was prepared from Intermediate N and Intermediate K using a similar methodology as described for the compound of Example 6. The final stage Boc deprotection was achieved by using 4M HCl in dioxane at 0° C. LCMS purity 92%, m/z 432 [M+H]⁺, ¹H NMR (400 MHz, CD₃OD) δ: 8.05 (1H, d, J=1.9 Hz), 7.65 (1H, d, J=2.1 Hz), 7.64 (1H, s), 3.56 (1H, t, J=6.1 Hz), 3.25 (3H, s), 2.64 (2H, t, J=7.3 Hz), 2.30 (3H, s), 2.14-2.04 (2H, m).

### Example 16

### Cyclopentyl N-[3-({5-[4-chloro-3-(methylsulfonyl)phenyl]-4-methyl-1,3-thiazol-2-yl}amino)-3-oxopropyl]-L-alaninate

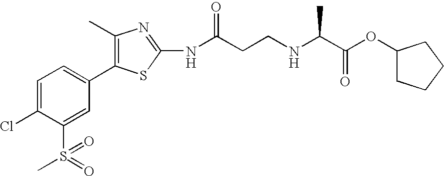

Example 16 was prepared from Intermediate N and Intermediate I using a similar methodology as described for the compound of Example 6 starting with 4-bromopropionic acid benzyl ester. LCMS purity 95%, m/z 514 [M+H]⁺, ¹H NMR (400 MHz, CD₃OD) δ: 8.15 (1H, d, J=1.9 Hz), 7.80-7.70 (2H, m), 5.37-5.29 (1H, m), 4.15 (1H, q, J=7.2 Hz), 3.57-3.39 (2H, m), 3.36 (3H, s), 3.02 (2H, t, J=6.2 Hz), 2.42 (3H, s), 2.02-1.62 (9H, m), 1.59 (3H, d, J=7.2 Hz).

### Example 17

### Cyclopentyl N-[3-({5-[4-chloro-3-(methylsulfonyl)phenyl]-4-methyl-1,3-thiazol-2-yl}amino)propyl]-L-leucinate

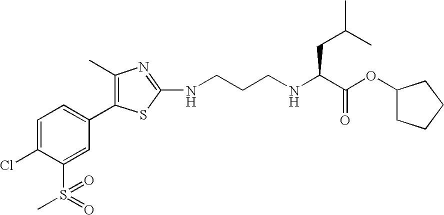

Example 17 was prepared by the following methodology

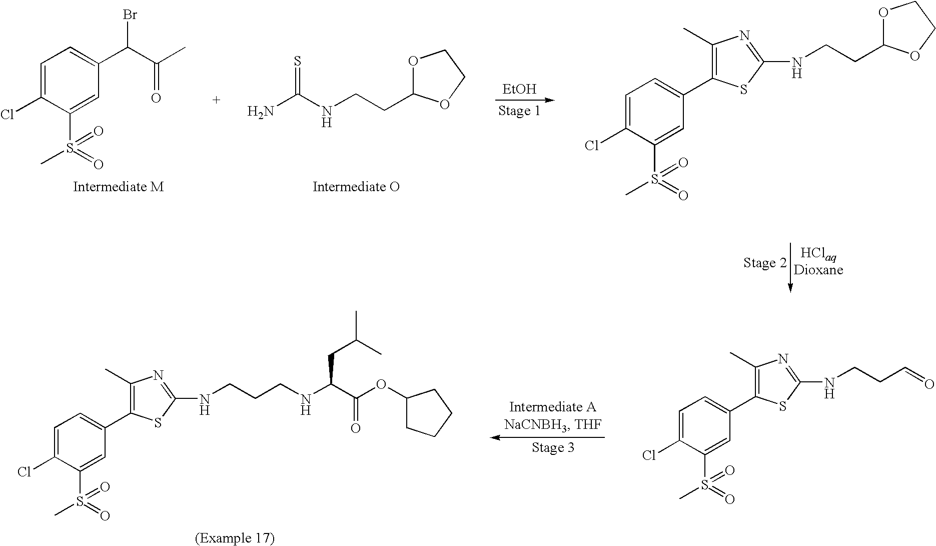

### Stage 1

A solution of Intermediate O (0.244 g, 1.39 mmol) in EtOH (10 ml) was added in one portion at RT to a pale yellow solution of Intermediate M (0.451 g, 1.39 mmol) in EtOH (15 ml). The resultant solution was stirred at 70° C. for 1 h. Upon cooling to RT the solid formed was isolated by filtration and washed with EtOH and then TBME (0.51 g, 91%). m/z 403/405 [M+H]⁺, ¹H NMR (400 MHz, CD₃OD) δ: 2.05-2.15 (2H, m), 2.35 (3H, s), 3.35 (3H, s), 3.60-3.65 (2H, m), 3.85-4.05 (4H, m), 5.00-5.05 (1H, m), 7.75-7.85 (2H, m), 8.15 (1H, s).

### Stage 2

2M HCl_{aq} (20 ml) was added to a suspension of stage 1 product (0.51 g, 1.27 mmol) in 1,4-dioxane (40 ml) at RT. The resultant suspension was stirred at RT for 1 h. The reaction mixture was gently warmed with a heat gun giving a colourless solution which was stirred for a further 1 h at RT. The reaction mixture was neutralised with sat NaHCO_{3(aq)} slowly and extracted with EtOAc (3×40 ml). The combined organic phase was washed with water (50 ml), dried (Na₂SO₄), filtered and concentrated in vacuo to afford the desired compound (0.424 g, 94%). m/z 359/361 [M+H]⁺, ¹H NMR (400 MHz, CDCl₃) δ: 2.25 (3H, s), 2.75-2.85 (2H, m), 3.25 (3H, s), 3.55 (2H, m), 7.40-7.50 (2H, m), 8.00 (1H, s), 9.85 (1H, br s).

### Stage 3

To a mixture of stage 2 product (50 mg, 0.04 mmol) and Intermediate A (41.8 mg, 0.21 mmol) in THF (3 ml) was added AcOH glacial dropwise (˜2 drops) until pH 5-6. The reaction mixture was stirred at RT for 30 min before adding NaCNBH₃ (35 mg, 0.56 mmol). Stirring was continued at RT for 18 h. The reaction mixture was evaporated to dryness by blowing under N₂, redissolved in EtOAc (7 ml) and washed with sat NaHCO_{3(aq)} (3 ml), dried (Na₂SO₄), filtered and concentrated in vacuo. Purification by preparative HPLC afforded the title compound (22 mg, 24%). LCMS purity 100%, m/z 542/544 [M+H]⁺, ¹H NMR (400 MHz, d₆-DMSO) δ: 0.95-1.05 (6H, m), 1.65-2.05 (11H, m), 2.10-2.20 (2H, m), 2.35 (3H, s), 3.10-3.25 (2H, m), 3.35 (3H, s, masked by DMSO peak), 3.55-3.65 (2H, m), 4.00-4.10 (1H, m), 5.35-5.40 (1H, m), 7.70-7.80 (2H, m), 8.15 (1H, s).

### Example 18

### Cyclopentyl N-{4-[({5-[4-chloro-3-(methylsulfonyl)phenyl]-4-methyl-1,3-thiazol-2-yl}amino)methyl]cyclohexyl}-L-leucinate

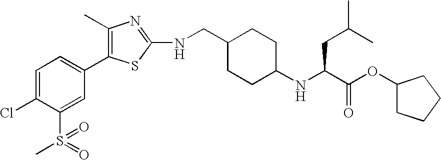

Example 18 was prepared from Intermediate M, Intermediate P and Intermediate A using a similar methodology as described for the compound of Example 17. LCMS purity 97%, m/z 596/598 [M+H]⁺, ¹H NMR (400 MHz, CD₃OD) δ: 0.80-0.95 (6H, m), 1.25 (9H, s), 1.45-1.80 (11H, m), 2.30 (3H, s), 2.55-2.65 (1H, m), 3.20-3.30 (2H, m), 3.35 (3H, s, masked by MeOD peak), 5.15-5.25 (1H, m), 7.65-7.70 (2H, m), 8.05 (1H, s).

### Example 19

### tert-Butyl N-{4-[({5-[4-chloro-3-(methylsulfonyl)phenyl]-4-methyl-1,3-thiazol-2-yl}amino)methyl]cyclohexyl}-L-leucinate

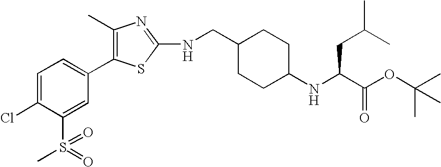

Example 19 was prepared from Intermediate M, Intermediate P and Intermediate J using a similar methodology as described for Example 17. LCMS purity 98%, m/z 584/586 [M+H]⁺, ¹H NMR (400 MHz, CD₃OD) δ: 0.85-1.00 (6H, m), 1.25-1.80 (21H, m), 2.35 (3H, s), 2.60 (1H, br s), 3.15-3.25 (2H, m, masked by MeOD peak), 3.35 (3H, s, masked by MeOD peak), 4.85 (1H, m, masked by H₂O peak), 7.60-7.65 (2H, m), 8.05 (1H, s).

### Example 20

### Cyclopentyl N-{[5-(2-acetamido-4-methyl-1,3-thiazol-5-yl)-2-chlorophenyl]sulfonyl}-L-alaninate

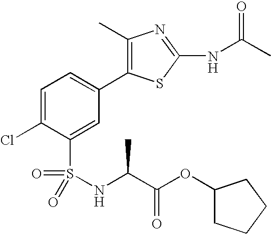

Example 20 was prepared by the following methodology

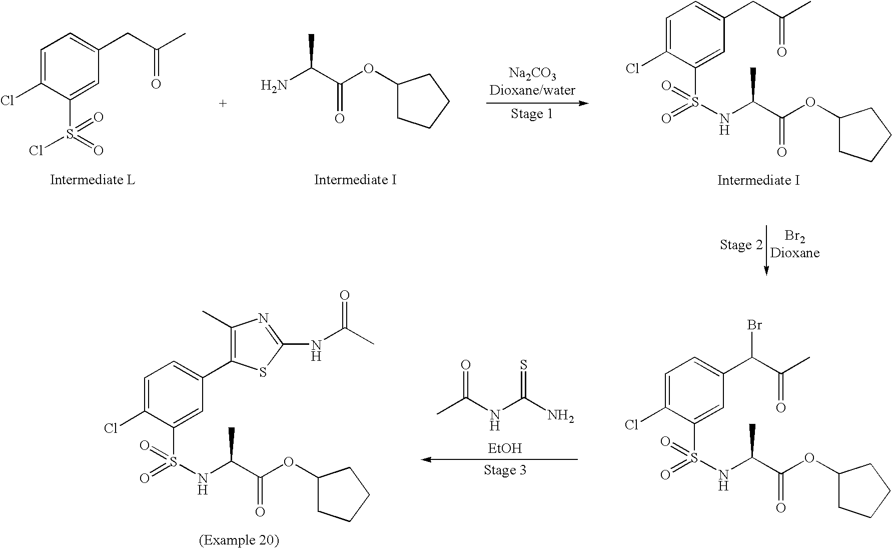

### Stage 1

To a solution of Intermediate L (800 mg, 3 mmol) in 1,4-dioxane (20 ml) was added Na₂CO₃ (636 mg, 6 mmol) in water (3 ml) followed by Intermediate I (472 mg, 3 mmol). The reaction was stirred at RT for 3 h. The reaction mixture was diluted with EtOAc (50 ml) and washed with water (150 ml) then brine (100 ml), dried (MgSO₄) and concentrated in vacuo to afford a brown oil (740 mg, 64%). This was used directly in the next stage without further purification. m/z 388/390 [M+H]⁺, ¹H NMR (300 MHz, CDCl₃) δ: 1.19 (3H, t), 1.27 (2H, d), 1.98 (6H, s), 2.16 (2H, s), 4.05 (3H, q), 4.96 (1H, m), 5.68 (1H, d), 7.26 (1H, dd), 7.42 (1H, d), 7.79 (1H, d).

### Stage 2

Stage 1 product (740 mg, 1.9 mmol) was dissolved in 1,4-dioxane (15 ml) and slowly treated with bromine (0.73 ml, 1.43 mmol). The reaction was stirred at RT for 1.5 h. The solvent was then concentrated in vacuo (maintaining the bath temp below 20° C.). The residue was dissolved in EtOAc (50 ml), washed with sat NaHCO_{3(aq)} (50 ml) then brine (50 ml), dried (MgSO₄) and concentrated in vacuo to afford the desired product as an orange oil (691 mg, 78%). This was used directly in the next stage without further purification. m/z 466/468 [M+H]⁺, ¹H NMR (300 MHz, CDCl₃) δ: 0.82 (2H, d), 1.09-1.27 (11H, m), 2.33 (2H, d), 4.93 (1H, m), 5.30 (1H, d), 5.68 (1H, m), 7.47 (1H, m), 7.63 (1H, m), 7.98 (1H, m).

### Stage 3

Stage 2 product (685 mg, 1.5 mmol) was dissolved in EtOH (25 ml) and treated with acetylthiourea (177 mg, 1.5 mmol). The reaction was stirred at 70° C. for 1.5 h. Upon cooling to RT a precipitate formed. This was isolated by filtration and washed with a small amount of ice-cold EtOH. The resulting brown solid was purified by prep HLPC (MeCN/water) to afford the title compound as white solid (25 mg, 3%). LCMS purity 100%, m/z 486 [M+H]⁺, ¹H NMR (300 MHz, CD₃OD) δ: 8.05 (1H, t, J=1.3 Hz), 7.63 (2H, d, J=1.3 Hz), 4.01 (1H, d, J=7.2 Hz), 2.38 (3H, s), 2.21 (3H, s), 1.45-1.79 (9H, m), 1.36 (3H, d, J=7.2 Hz).

### Example 21

### Cyclopentyl N-{[5-(2-acetamido-4-methyl-3-thiazol-5-yl)-2-chlorophenyl]sulfonyl}-O-tert-butyl-L-serinate

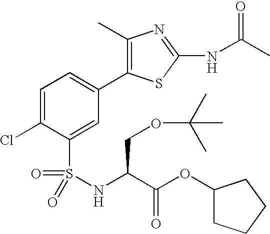

Example 21 was prepared from Intermediate L and Intermediate F using a similar methodology as described for the compound of Example 20. LCMS purity 100%, m/z 558 [M+H]⁺, ¹H NMR (300 MHz, CD₃OD) δ: 8.08 (1H, s), 7.65 (2H, s), 4.08-4.15 (1H, m), 2.39 (3H, s), 2.23 (3H, s), 1.70-1.81 (2H, m), 1.47 (9H, m), 1.10 (9H, s).

### Example 22

### Cyclopentyl N-[2-({[5-(2-acetamido-4-methyl-1,3-thiazol-5-yl)-2-chlorophenyl]sulfonyl}amino)ethyl]-L-leucinate

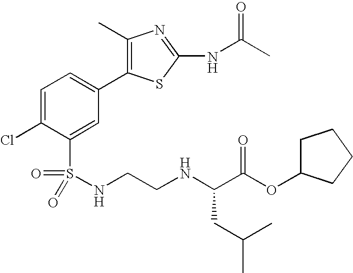

Example 22 was prepared by the following methodology

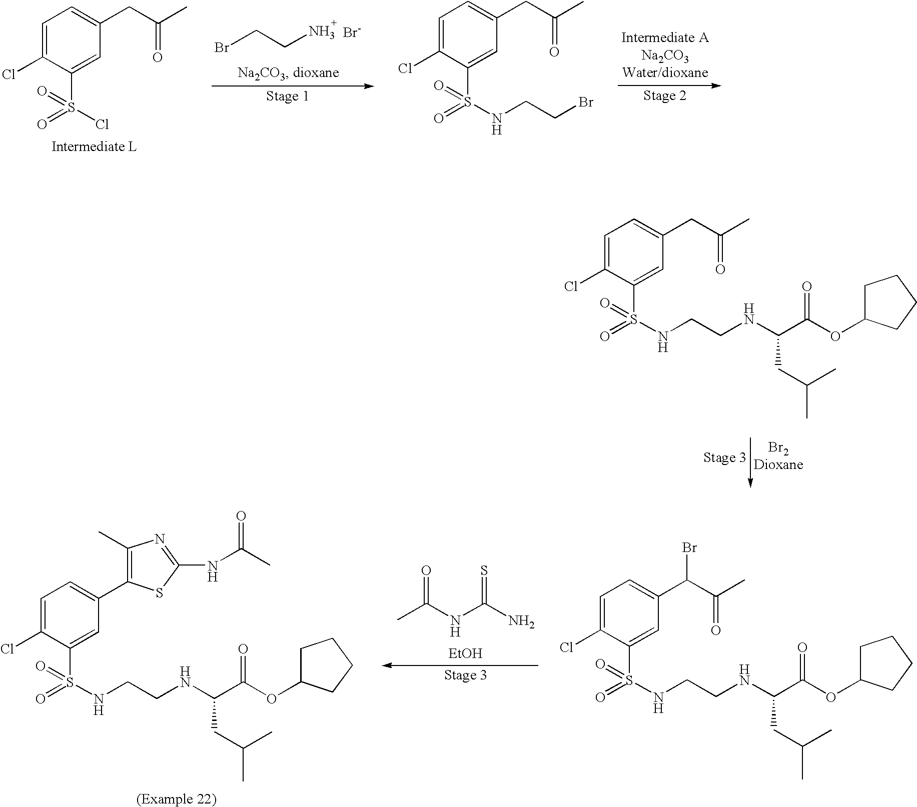

### Stage 1

To a solution of Intermediate L (760 mg, 2.85 mmol) in 1,4-dioxane (15 ml) was added Na₂CO₃ (603 mg, 5.7 mmol) followed by 2-bromoethylamine HBr salt (584 mg, 2.85 mmol). The reaction was stirred at RT for 1 h. The solvent was removed in vacuo and the residue re-dissolved in EtOAc (50 ml) and washed with water (20 ml) then dried (MgSO₄) and concentrated to give a brown foam (808 mg, 80%). This was used directly in the next stage without further purification. m/z 354 [M+H]⁺, ¹H NMR (300 MHz, CDCl₃) δ: 1.97 (3H, s), 3.55 (2H, t), 3.61 (2H, d), 4.37 (2H, t), 5.09 (1H, br s), 7.28 (1H, dd), 7.43 (1H, d), 7.81 (1H, d).

### Stage 2

To a solution of stage 1 product (400 mg, 1.13 mmol) in 1,4-dioxane (8 ml) was added Na₂CO₃ (240 mg, 2.26 mmol) in water (2 ml), followed by Intermediate A (267 mg, 1.13 mmol). The reaction was stirred at RT for 36 h. The solvent was removed in vacuo and the resulting residue dissolved in EtOAc (50 ml) and washed with water (50 ml), dried (MgSO₄) and concentrated. The crude was purified by flash chromatography (30% EtOAc in heptane) to afford the desired product (64 mg, 12%). m/z 474 [M+H]⁺, ¹H NMR (300 MHz, CDCl₃) δ: 1.28-1.81 (14H, m), 2.25 (3H, s), 2.48 (1H, m), 2.77-2.89 (2H, m), 2.99-3.11 (2H, m), 3.79 (2H, s), 5.20 (1H, m), 5.79 (1H, t), 7.36 (1H, dd), 7.50 (1H, d), 7.92 (1H, d).

### Stage 3

Stage 2 product (63 mg, 0.133 mmol) was dissolved in 1,4-dioxane (10 ml) and treated slowly with bromine (5 μl, 0.1 mmol). The reaction was stirred at RT for 76 h. The solvent was removed in vacuo (maintaining the bath temp below 25° C.) and the residue dissolved in EtOAc (15 ml). This was washed with water (20 ml) sat NaHCO_{3(aq)} (20 ml) and brine (20 ml) then dried (MgSO₄) and concentrated to afford an orange oil (72 mg, 98%). This was used directly in the next stage without further purification. m/z 551/553 [M+H]⁺.

### Stage 4

Stage 3 product (70 mg, 0.13 mmol) and acetylthiourea (15 mg, 0.13 mmol) were dissolved in EtOH (3 ml) and heated to 70° C. for 1.5 h. The solvent was removed in vacuo and the residue purified by prep HPLC (MeCN/0.05% TFA_{aq}) to afford the title compound as a cream coloured solid (26 mg, 39%). LCMS purity 99%, m/z 571 [M+H]⁺, ¹H NMR (300 MHz, CD₃OD) δ: 8.11 (1H, s), 7.22 (2H, s), 5.33 (1H, t, J=5.5 Hz), 4.11 (1H, t, J=4.4 Hz), 3.20-3.27 (4H, m), 2.41 (3H, s), 2.23 (3H, s), 1.88-1.98 (2H, m), 1.66-1.84 (9H, m), 1.02 (6H, t, J=6.3 Hz).

### Example 23

### Cyclopentyl N-({5-[4-chloro-3-(methylsulfonyl)phenyl]-4-methyl-1,3-thiazol-2-yl}carbamoyl)-L-serinate

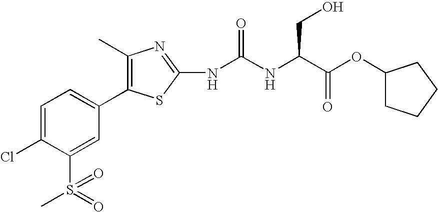

Example 23 was prepared by the following methodology

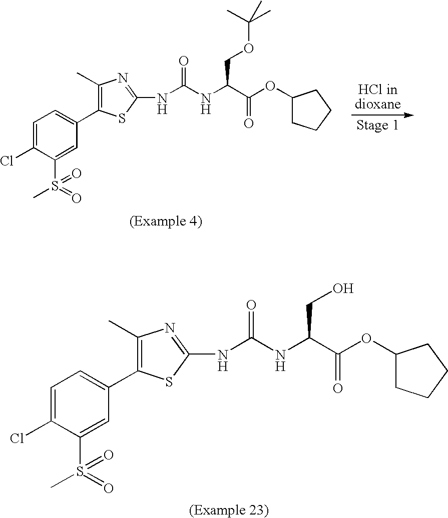

### Stage 1

The compound of Example 4 (32 mg, 0.06 mmol) was treated with 4M HCl in dioxane (5 ml) at 70° C. for 18 h. The solvent was removed in vacuo and the crude triturated with Et₂O/heptane to afford the title compound as a pale brown solid (5 mg, 16%). LCMS purity 85%, m/z 502/504 [M+H]⁺, ¹H NMR (300 MHz, CD₃OD) δ: 8.08 (1H, s), 7.72 (2H, s), 5.16 (1H, t, J=5.5 Hz), 4.36 (1H, t, J=3.3 Hz), 3.90 (1H, dd, J=10.9, 3.4 Hz), 3.79 (1H, dd, J=10.9, 3.4 Hz), 3.27 (3H, s), 2.36 (3H, s), 1.86-1.75 (2H, m), 1.73-1.62 (4H, m), 1.60-1.50 (2H, m).

### Example 24

### Cyclopentyl N-[4-({5-[4-chloro-3-(methylsulfonyl)phenyl]-4-methyl-1,3-thiazol-2-yl}amino)-4-oxobutyl]-L-serinate

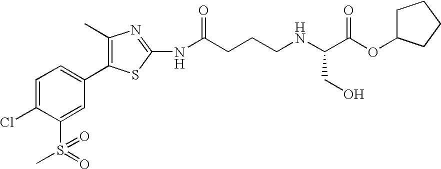

Example 24 was prepared from Example 12 using a similar methodology as described for the compound of Example 23. LCMS purity 95%, m/z 544 [M+H]⁺, ¹H NMR (300 MHz, CD₃OD) δ: 8.05 (1H, d, J=1.5 Hz), 7.67-7.64 (2H, m), 5.28-5.20 (1H, m), 4.07-4.01 (1H, m), 3.95 (2H, d, J=3.0 Hz), 3.66-3.44 (2H, m), 3.25 (3H, s), 2.63 (2H, t, J=6.7 Hz), 2.31 (3H, s), 2.07-2.00 (2H, m), 1.88-1.79 (2H, m), 1.76-1.50 (6H, m).

### Example 25

### Cyclopentyl N-{[5-(2-acetamido-4-methyl-1,3-thiazol-5-yl)-2-chloro phenyl]sulfonyl}-L-serinate

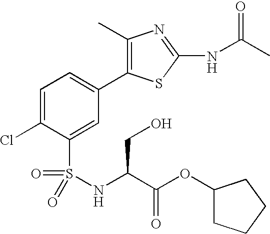

Example 25 was prepared from Example 21 using a similar methodology as described for the compound of Example 23. LCMS purity 100%, m/z 502 [M+H]⁺, ¹H NMR (300 MHz, CD₃OD) δ: 8.09 (1H, s), 7.66 (2H, s), 4.06 (1H, t, J=5.2 Hz), 2.40 (3H, s), 2.24 (3H, s), 1.69-1.79 (2H, m), 1.46-1.65 (9H, m), 1.10 (9H, m).

### Example 26

### Cyclopentyl N-[4-({5-[4-chloro-3-(methylsulfonyl)phenyl]-4-methyl-1,3-thiazol-2-yl}amino)-4-oxobutyl]-O-phosphono-L-serinate

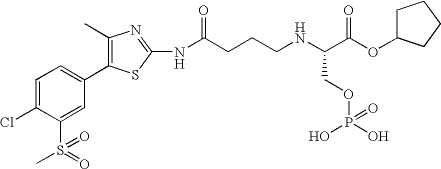

Example 26 was prepared by the following methodology

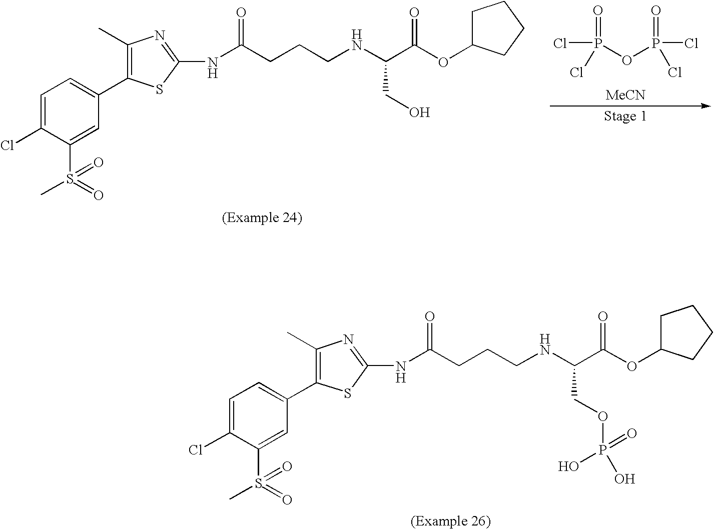

### Stage 1

To a solution of the compound of Example 24 (80 mg, 0.147 mmol) in MeCN (5 ml) at −5° C. was added pyrophosphorylchloride (81 μl, 0.588 mmol). The mixture was stirred at −5° C. for 2.5 h. The mixture was then poured onto ice-water (50 ml) and washed with EtOAc. The aqueous layer was basified to pH ˜7 with 2M NaOH_{aq} and then extracted with EtOAC (3×50 ml). As the product remained in the aqueous layer, the water was removed in vacuo. The resulting white solid was purified by prep HLPC (MeCN/0.05% TFA_{aq}) to afford the title compound as a clear oil (32 mg, 35%). LCMS purity 100%, m/z 624 [M+H]⁺, ¹H NMR (300 MHz, CD₃OD) δ: 8.16 (1H, d, J=1.6 Hz), 7.70-7.82 (2H, m), 5.33-5.41 (1H, m), 4.37-4.47 (2H, m), 4.34 (1H, br s), 3.36 (3H, s), 2.72 (2H, t, J=6.8 Hz), 2.41 (3H, s), 2.10-2.21 (2H, m), 1.71-2.02 (8H, m), 1.62-1.69 (2H, m).

### Example 27

### N-{5-[4-Chloro-3-(methylsulfonyl)phenyl]-4-methyl-1,3-thiazol-2-yl}-L-phenylalanine

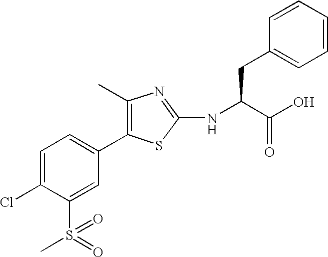

Example 27 was prepared by the following methodology

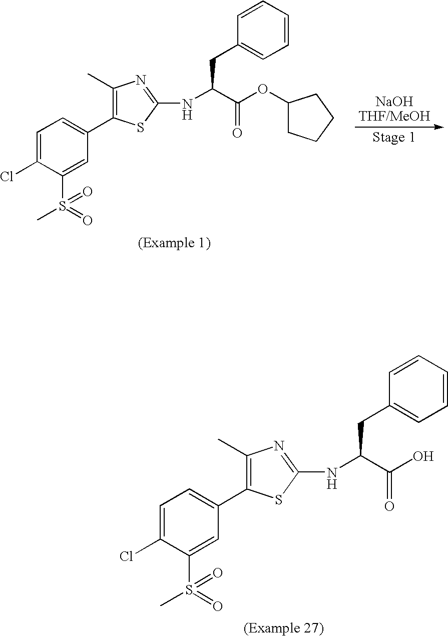

### Stage 1

To a solution of Example 1 (20 mg, 0.038 mmol) in a mixture of THF (0.5 ml) and MeOH (0.5 ml) was added 2M NaOH_{aq} (0.5 ml). The mixture was allowed to stand at RT for 1.5 h. Upon completion the reaction mixture was concentrated to near dryness, 1M HCl_{aq} was added dropwise until pH 5-6 and resulted in precipitate formation. The pale yellow solid was collected by filtration under slight pressure and dried in vacuo (12 mg, 70%). LCMS purity 99%, m/z 451 [M+H]⁺, ¹H NMR (400 MHz, CD₃OD) δ: 2.30 (3H, s), 3.05-3.15 (1H, m), 3.35 (3H, s, masked by MeOH peak), 3.35-3.45 (1H, m), 4.70-4.75 (1H, m), 7.20-7.35 (5H, m), 7.65-7.75 (2H, m), 8.05-8.10 (1H, m).

### Example 28

### 2S)-({5-[4-Chloro-3-(methylsulfonyl)phenyl]-4-methyl-1,3-thiazol-2-yl}amino)(phenyl)acetic acid

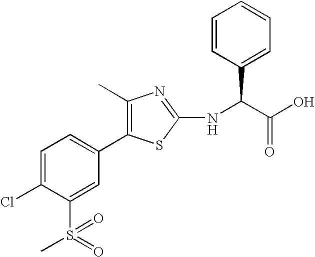

Example 28 was prepared from Example 2 using a similar methodology as described for the compound of Example 27. LCMS purity 99%, m/z 437 [M+H]⁺, ¹H NMR (400 MHz, CD₃OD) δ: 2.30 (3H, s), 3.35 (3H, s, masked by MeOH peak), 3.35-3.45 (1H, m), 5.50-5.55 (1H, m), 7.35-7.45 (3H, m), 7.50-7.60 (2H, m), 7.65-7.75 (2H, m), 8.05-8.10 (1H, m).

### Example 29

### N-{5-[4-Chloro-3-(methylsulfonyl)phenyl]-4-methyl-1,3-thiazol-2-yl}-L-leucine

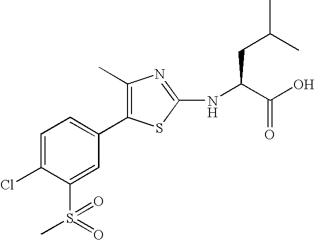

Example 29 was prepared from Example 3 using a similar methodology as described for the compound of Example 27. LCMS purity 92%, m/z 417 [M+H]⁺, ¹H NMR (400 MHz, CD₃OD) δ: 0.95-1.05 (6H, m), 1.70-1.90 (3H, m), 2.30 (3H, s), 3.35 (3H, s, masked by MeOH peak), 4.40-4.50 (1H, m), 7.65-7.75 (2H, m), 8.05-8.15 (1H, m).

### Example 30

### O-tert-Butyl-N-({5-[4-chloro-3-(methylsulfonyl)phenyl]-4-methyl-1,3-thiazol-2-yl}carbamoyl)-L-serine

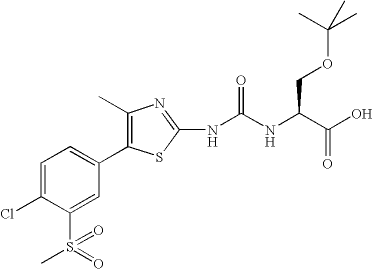

Example 30 was prepared from Example 4 using a similar methodology as described for the compound of Example 27. LCMS purity 97%, m/z 490/492 [M+H]⁺, ¹H NMR (300 MHz, CD₃OD) δ: 8.15 (1H, d, J=1.9 Hz), 7.74 (1H, d, J=2.1 Hz), 7.72 (1H, s), 4.28 (1H, t, J=3.4 Hz), 3.87-3.80 (1H, m), 3.76-3.70 (1H, m), 3.35 (3H, s), 2.38 (3H, s), 1.21 (9H, s).

### Example 31

### N-({5-[4-Chloro-3-(methylsulfonyl)phenyl]-4-methyl-1,3-thiazol-2-yl}carbamoyl)-L-phenylalanine

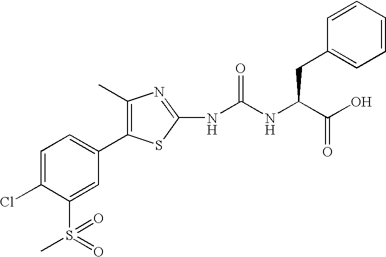

Example 31 was prepared from Example 5 using a similar methodology as described for the compound of Example 27. LCMS purity 100%, m/z 494/496 [M+H]⁺, ¹H NMR (400 MHz, CD₃OD) δ: 2.35 (3H, s), 3.05-3.20 (2H, m), 3.35 (3H, s, masked by MeOH peak), 4.65-4.75 (1H, m), 7.15-7.35 (5H, m), 7.75-7.80 (2H, m), 8.15 (1H, s).

### Example 32

### N-4-({5-[4-Chloro-3-(methylsulfonyl)phenyl-4-methyl-1,3-thiazol-2-yl}amino)-4-oxobutyl]-L-leucine

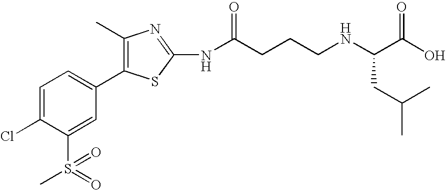

Example 32 was prepared from Example 6 using a similar methodology as described for the compound of Example 27. LCMS purity 92%, m/z 570/572 [M+H]⁺, ¹H NMR (400 MHz, CD₃OD) δ: 1.15-1.25 (6H, m), 1.80-1-95 (1H, m), 1.95-2.05 (2H, m), 2.20-2.30 (2H, m), 2.55 (3H, s), 2.80-2.90 (2H, m), 3.30-3.35 (2H, m), 3.50 (3H, s), 4.10-4.20 (1H, m), 7.85-7.95 (2H, m), 8.30 (1H, s).

### Example 33

### N-[4-({5-[4-Chloro-3-(methylsulfonyl)phenyl]-4-methyl-1,3-thiazol-2-yl}amino)-4-oxobutyl]-L-glutamic acid

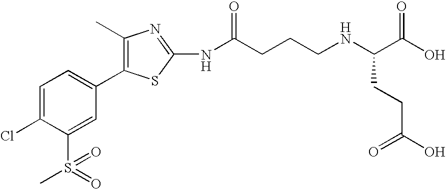

Example 33 was prepared from Example 8 using a similar methodology as described for the compound of Example 27. LCMS purity 92%, m/z 518 [M+H]⁺.

### Example 34

### (2R)-{[4-({5-[4-Chloro-3-(methylsulfonyl)phenyl]-4-methyl-1,3-thiazol-2-yl}amino)-4-oxobutyl]amino}(phenyl)acetic acid

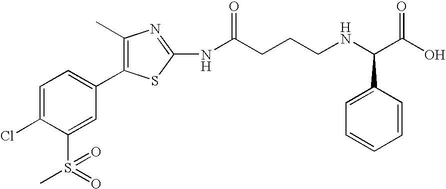

Example 34 was prepared from Example 9 using a similar methodology as described for the compound of Example 27. LCMS purity 95%, m/z 522/524 [M+H]⁺, ¹H NMR (400 MHz, CD₃OD) δ: 1.90-2.20 (2H, m), 2.35 (3H, s), 2.60-2.70 (2H, m), 2.95-3.20 (2H, m), 3.35 (3H, s), 5.15-5.20 (1H, m), 7.45-7.55 (5H, m), 7.75-7.80 (2H, m), 8.15 (1H, s).

### Example 35

### (2S)-{[4-({5-[4-Chloro-3-(methylsulfonyl)phenyl]-4-methyl-1,3-thiazol-2-yl}amino)-4-oxobutyl]amino}(phenyl)acetic acid

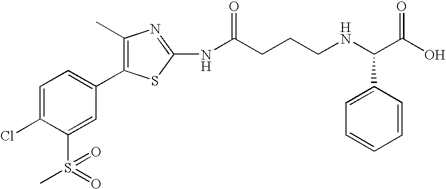

Example 35 was prepared from Example 10 using a similar methodology as described for the compound of Example 27. LCMS purity 96%, m/z 522/524 [M+H]⁺, ¹H NMR (400 MHz, CD₃OD) δ: 2.00-2.20 (2H, m), 2.35 (3H, s), 2.60-2.70 (2H, m), 2.95-3.20 (2H, m), 3.35 (3H, s), 5.15-5.20 (1H, m), 7.50-7.60 (5H, m), 7.75-7.80 (2H, m), 8.15 (1H, s).

### Example 36

### N-[4-({5-[4-Chloro-3-(methylsulfonyl)phenyl]-4-methyl-1,3-thiazol-2-yl}amino)-4-oxobutyl]-L-phenylalanine

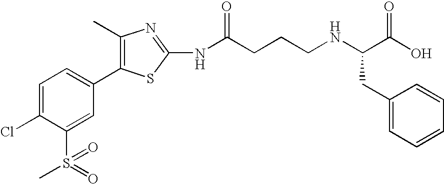

Example 36 was prepared from Example 11 using a similar methodology as described for the compound of Example 27. LCMS purity 97%, m/z 536/538 [M+H]⁺, ¹H NMR (400 MHz, CD₃OD) δ: 1.95-2.05 (2H, m), 2.35 (3H, s), 2.55-2.65 (2H, m), 3.05-3.15 (2H, m), 3.34 (2H, m, masked by MeOH peak), 3.40 (3H, s), 4.20-4.25 (1H, m), 7.15-7.30 (5H, m), 7.65-7.70 (2H, m), 8.05 (1H, s).

### Example 37

### O-tert-Butyl-N-[4-({5-[4-chloro-3-(methylsulfonyl)phenyl]-4-methyl-1,3-thiazol-2-yl}amino)-4-oxobutyl]-L-serine

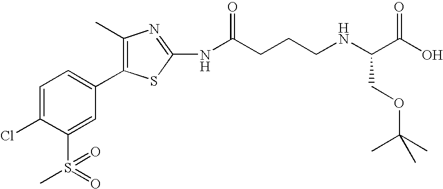

Example 37 was prepared from Example 12 using a similar methodology as described for the compound of Example 27. LCMS purity 95%, m/z 532 [M+H]⁺, ¹H NMR (300 MHz, CD₃OD) δ: 8.16 (1H, d, J=1.7 Hz), 7.77-7.75 (2H, m), 3.88 (1H, d, J=3.6 Hz), 3.83-3.76 (1H, m), 3.69-3.64 (1H, m), 3.37 (3H, s), 3.27-3.22 (2H, m), 2.75-2.69 (2H, m), 2.42 (3H, s), 2.18-2.07 (2H, m), 1.26 (9H, s).

### Example 38

### N-[3-({5-[4-Chloro-3-(methylsulfonyl)phenyl]-4-methyl-1,3-thiazol-2-yl}amino)-3-oxopropyl]-L-alanine

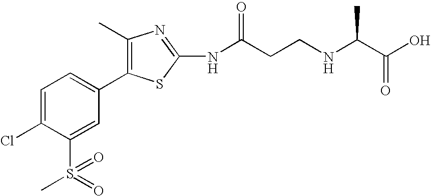

Example 38 was prepared from Example 16 using a similar methodology as described for the compound of Example 27. LCMS purity 95%, m/z 446 [M+H]⁺, ¹H NMR (300 MHz, d₆-DMSO) δ: 8.01 (1H, s), 7.85-7.77 (2H, m), 3.30-3.21 (3H, m), 3.17-2.98 (4H, m), 2.86 (2H, t, J=6.4 Hz), 2.38 (3H, s), 1.29 (3H, d, J=7.0 Hz). Note: NMR integration not accurate because of strong water peak present.

### Example 39

### N-[3-({5-[4-Chloro-3-(methylsulfonyl)phenyl]-4-methyl-1,3-thiazol-2-yl}amino)propyl]-L-leucine

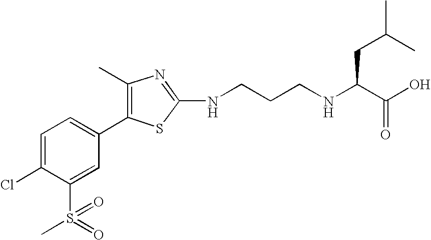

Example 39 was prepared from Example 17 using a similar methodology as described for the compound of Example 27. LCMS purity 90%, m/z 474/476 [M+H]⁺, ¹H NMR (400 MHz, CD₃OD) δ: 0.95-1.05 (6H, m), 1.65-1.75 (1H, m), 1.75-1.95 (2H, m), 2.15-2.20 (2H, m), 2.35 (3H, s), 3.15-3.25 (2H, m), 3.35 (3H, s, masked by MeOD peak), 3.45-3.60 (2H, m), 3.95-4.05 (1H, m), 7.70-7.75 (2H, m), 8.10 (1H, s).

### Example 40

### N-{4-[({5-[4-Chloro-3-(methylsulfonyl)phenyl]-4-methyl-1,3-thiazol-2-yl}amino)methyl]cyclohexyl}-L-leucine

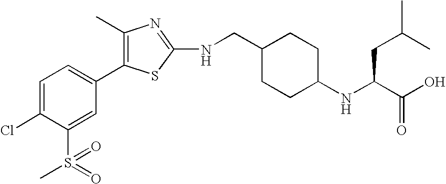

Example 40 was prepared from Example 18 using a similar methodology as described for the compound of Example 27. LCMS purity 90%, m/z 528/530 [M+H]⁺, ¹H NMR (400 MHz, CD₃OD) δ: 0.85-1.00 (6H, m), 1.65-1.80 (9H, m), 1.85-1.95 (2H, m), 2.00-2.15 (1H, m), 2.30 (3H, s), 3.20 (1H, m, masked by MeOD peak), 3.30 (3H, s), 3.35-3.45 (2H, m), 3.95-4.00 (1H, m), 7.65-7.75 (2H, m), 8.05 (1H, s).

### Example 41

### N-{[5-(2-Acetamido-4-methyl-1,3-thiazol-5-yl)-2-chlorophenyl]sulfonyl}-L-alanine

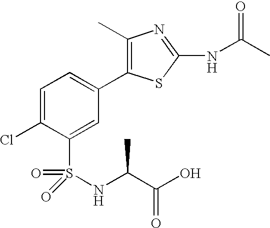

Example 41 was prepared from Example 20 using a similar methodology as described for the compound of Example 27. LCMS purity 98%, m/z 418 [M+H]⁺, ¹H NMR (300 MHz, CD₃OD) δ: 8.07 (1H, s), 7.63 (2H, d, J=1.0 Hz), 3.57 (1H, d, J=6.9 Hz), 2.39 (3H, s), 2.22 (3H, s), 1.34 (3H, d, J=7.0 Hz).

### Example 42

### N-{[5-(2-Acetamido-4-methyl-1,3-thiazol-5-yl)-2-chlorophenyl]sulfonyl}-O-tert-butyl-L-serine

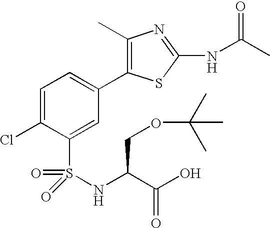

Example 42 was prepared from Example 21 using a similar methodology as described for the compound of Example 27. LCMS purity 100%, m/z 490 [M+H]⁺, ¹H NMR (300 MHz, CD₃OD) δ: 8.09 (1H, s), 7.63 (2H, s), 4.15 (1H, t, J=4.3 Hz), 3.68 (1H, dd), 3.56 (1H, dd, J=9.3, 4.2 Hz), 2.38 (3H, s), 2.22 (3H, s), 1.08 (9H, s).

### Example 43

### N-[2-({[5-(2-Acetamido-4-methyl-1,3-thiazol-5-yl)-2-chlorophenyl]sulfonyl}amino)ethyl]-L-leucine

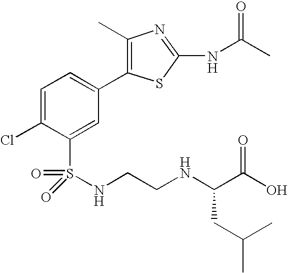

Example 43 was prepared from Example 22 using a similar methodology as described for the compound of Example 27. LCMS purity 100%, m/z 503 [M+H]⁺, ¹H NMR (300 MHz, d₆-DMSO) δ: 7.95 (1H, s), 7.71 (2H, s), 2.91-3.10 (4H, m), 2.81 (1H, t, J=6.9 Hz), 2.36 (3H, s), 2.16 (3H, s), 2.08 (1H, s), 1.82 (1H, s), 0.80 (7H, dd, J=9.4, 6.6 Hz).

### Example 44

### N-{[5-(2-Acetamido-4-methyl-1,3-thiazol-5-yl)-2-chlorophenyl]sulfonyl}-L-serine

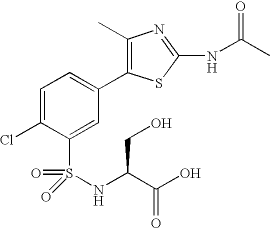

Example 44 was prepared from the compound of Example 25 using a similar methodology as described for Example 27. LCMS purity 100%, m/z 434 [M+H]⁺, ¹H NMR (300 MHz, CD₃OD) δ: 12.20 (1H, s), 8.16 (1H, d, J=8.7 Hz), 8.02 (1H, s), 7.68 (1H, s), 3.90-3.98 (1H, m), 3.64 (2H, d, J=5.2 Hz), 2.37 (3H, s), 2.16 (3H, s).

### Example 45

### N-[4-({5-[4-Chloro-3-(methylsulfonyl)phenyl]-4-methyl-1,3-thiazol-2-yl}amino)-4-oxobutyl]-L-serine

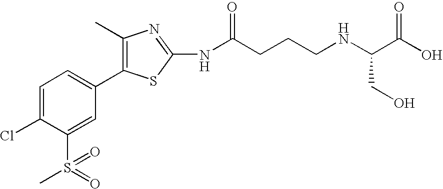

Example 45 was prepared by the following methodology

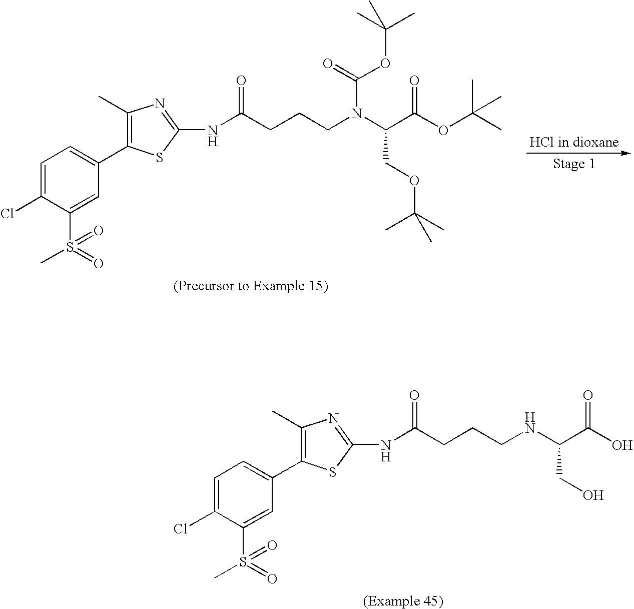

The precursor to Example 15 (prepared as described for the compound of Example 6-153 mg, 0.22 mmol) was treated with 4M HCl in dioxane (5 ml) and heated to 70° C. The reaction was stirred at 70° C. for 2 h. The solvent was then removed in vacuo and the resulting gum was triturated with Et₂O/heptane to afford the title compound as a white solid (80 mg, 76%). LCMS purity 95%, m/z 476 [M+H]⁺, ¹H NMR (300 MHz, d₆-DMSO) δ: 12.36 (1H, br s), 9.17 (1H, br s), 9.05 (1H, br s), 8.01 (1H, s), 7.82 (2H, s), 4.11-4.04 (1H, m), 4.00-3.84 (2H, m), 3.43 (3H, s), 3.19-3.15 (2H, m), 2.58 (2H, t, J=7.0 Hz), 2.39 (3H, s), 2.08-1.95 (2H, m).

### Biological Results

### (A) Broken Cell Carboxylesterase Assay

Any given compound of the present invention wherein R₁ is an ester group may be tested to determine whether it meets the requirement that it be hydrolysed by intracellular esterases, by testing in the following assay.

### Preparation of Cell Extract

U937 or Hut78 tumour cells (˜10⁹) were washed in 4 volumes of Dulbeccos PBS (˜1 litre) and pelleted at 525 g for 10 min at 4° C. This was repeated twice and the final cell pellet was resuspended in 35 ml of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:
Leupeptin 1 μM
Aprotinin 0.1 μM
E64 8 μM
Pepstatin 1.5 μM
Bestatin 162 μM
Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 ml) was incubated at 37° C. in a Tris-HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 min). Reactions were stopped by the addition of 3× volumes of acetonitrile. For zero time samples the acetonitrile was added prior to the ester compound. After centrifugation at 12000 g for 5 min, samples were analysed for the ester and its corresponding carboxylic acid at room temperature by LCMS (Sciex API 3000, HP1100 binary pump, CTC PAL). Chromatography was based on an AceCN (75×2.1 mm) column and a mobile phase of 5-95% acetonitrile in water/0.1% formic acid.

Rates of hydrolysis are expressed in pg/mL/min.

Table 1 presents data showing that several amino acid ester motifs, conjugated to various intracellular enzyme inhibitors by several different linker chemistries are all hydrolysed by intracellular carboxyesterases to the corresponding acid.

TABLE 1

| | | | Hydrolysis Rate Range | Preparation of amino |
|---|---|---|---|---|
| Structure of amino acid ester conjugate | R | Linker | U937Cells (pg/mL/min) | ester conjugate |
| 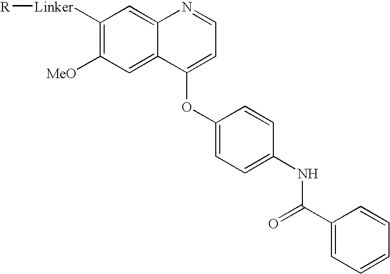 | 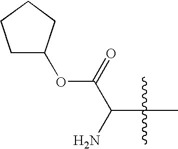 | —CH2CH2O— | 100-1000 | WO2006117552 |
| 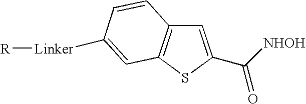 | 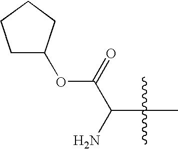 | 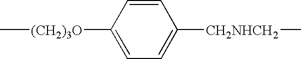 | 1000-50000 | WO2006117548 |
| 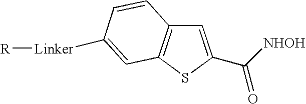 | 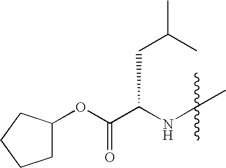 | 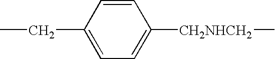 | >50000 | WO2006117549 |
| 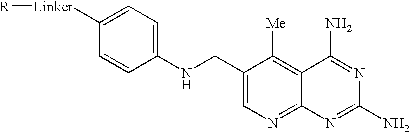 | 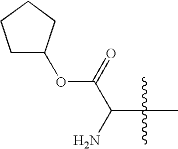 | —CH2CH2O— | >50000 | WO2006117567 |
| 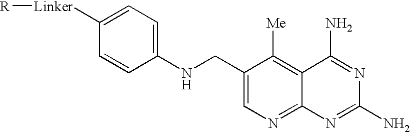 | 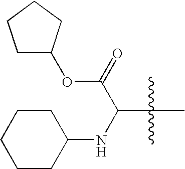 | —CH2CH2O— | 1000-50000 | WO2006117567 |
| 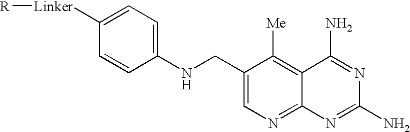 | 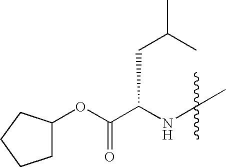 | —CH2— | 1000-50000 | WO2006117567 |
| 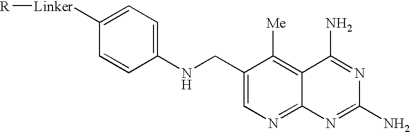 | 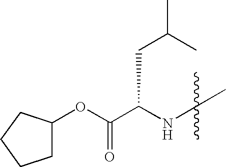 | —CO— | >50000 | WO2006117567 |
| 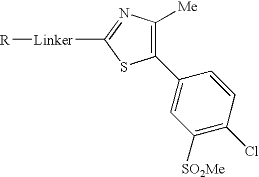 | 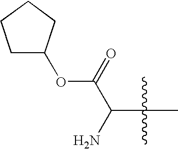 | —CH2CH2CONH— | 100-1000 | |
| 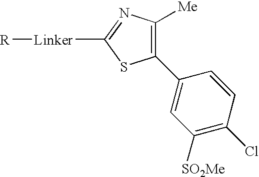 | 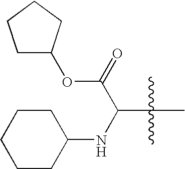 | —CH2CH2CONH— | 100-1000 | |
| 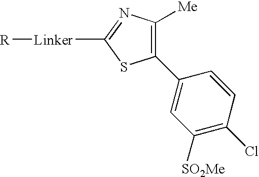 | 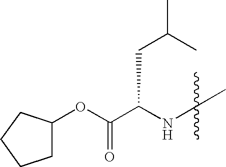 | 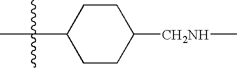 | 1000-50000 | |
| 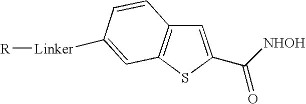 | 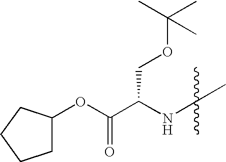 | 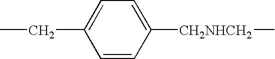 | >50000 | WO2006117549 |
| 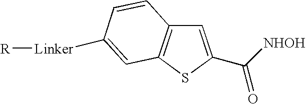 | 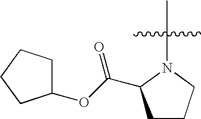 | 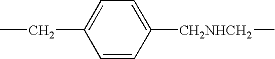 | >50000 | WO2006117549 |

### (B) Inhibition of PI3 Kinase V Activity

In a final reaction volume of 20 μl, PI3Kγ (human) is incubated in assay buffer containing 10 μM phosphatidylinositol-4,5-bisphosphate and MgATP (concentration as required). The reaction is initiated by the addition of the MgATP mix. After incubation for 30 minutes at room temperature, the reaction is stopped by the addition of 5 μl of stop solution containing EDTA and biotinylated phosphatidylinositol-3,4,5-trisphosphate. Finally, 5 μl of detection buffer is added, which contains europium-labelled anti-GST monoclonal antibody, GST-tagged GRP1 PH domain and streptavidin-allophycocyanin. The plate is then read in time-resolved fluorescence mode and the homogenous time-resolved fluorescence (HTRF) signal is determined according to the formula HTRF=10000×(Em665 nm/Em620 nm).

Duplicate data points are generated from a ⅓ log dilution series of a stock solution of compound in DMSO. Nine dilutions steps are made from a top concentration of 10 μM, and a ‘no compound’ blank is included. The HTRF PI 3-Kinase assay is performed at an ATP concentration at, or close to, the Km. HTRF ratio data is transformed into % activity of controls and analysed with a four parameter sigmoidal dose-response (variable slope) application. QC criteria is based on Top, Bottom, Hill slope, r² and IC50, the concentration giving 50% inhibition, which is reported.

### (C) LPS-Stimulation of THP-1 Cells

THP-1 cells are plated in 100 μl at a density of 4×10⁴ cells/well in V-bottomed 96 well tissue culture treated plates and incubated at 37° C. in 5% CO₂ for 16 h. 2 h after the addition of the inhibitor in 100 μl of tissue culture media, the cells are stimulated with LPS (E. coli strain 005:B5, Sigma) at a final concentration of 1 μg/ml and incubated at 37° C. in 5% CO₂ for 6 h. TNF-α levels are measured from cell-free supernatants by sandwich ELISA (R&D Systems #QTA00B)

### (D) LPS-Stimulation of Human Whole Blood

Whole blood is taken by venous puncture using heparinised vacutainers (Becton Dickinson) and diluted in an equal volume of RPMI1640 tissue culture media (Sigma). 100 μl is plated in V-bottomed 96 well tissue culture treated plates. 2 h after the addition of the inhibitor in 100 μl of RPMI1640 media, the blood is stimulated with LPS (E. coli strain 005:B5, Sigma) at a final concentration of 100 ng/ml and incubated at 37° C. in 5% CO₂ for 6 h. TNF-α levels are measured from cell-free supernatants by sandwich ELISA (R&D Systems #QTA00B)

For each of the assays (B), (C) and (D) above, IC50 values are allocated to one of three ranges as follows:

### Range A: IC50<100 nM

Range B: 100 nM<IC50<1000 nM

### Range C: IC50>1000 nM

The results are as shown in Table 2. Blank cells in Table 2 indicate that the compound was not tested at the date of the present application.

TABLE 2

| | Inhibitor | Inhibitor activity | Inhibitor activity versus |
|---|---|---|---|
| | activity versus | versus THP-1 | human whole blood |
| Example | PI3 Kinase γ | TNFα release | TNFα release |
| 4 | B | C | |
| 6 | B | B | C |
| 7 | C | B | B |
| 8 | B | | |
| 9 | B | C | |
| 10 | B | C | |
| 11 | C | C | |
| 12 | C | C | |
| 13 | C | C | |
| 14 | C | C | |
| 15 | C | C | |
| 16 | C | B | |
| 17 | C | C | |
| 18 | C | C | |
| 19 | C | C | |
| 20 | A | C | |
| 21 | B | C | |
| 22 | C | C | |
| 23 | C | C | |
| 24 | C | A | B |
| 25 | B | C | |
| 26 | A | C | |
| 30 | C | | |
| 31 | C | | |
| 32 | C | | |
| 33 | C | | |
| 34 | C | | |
| 35 | C | | |
| 36 | C | | |
| 37 | C | | |
| 38 | C | | |
| 39 | C | | |
| 40 | C | | |
| 41 | C | | |
| 42 | C | | |
| 43 | C | | |
| 44 | C | | |
| 45 | C | | |

1. A compound of formula (I): 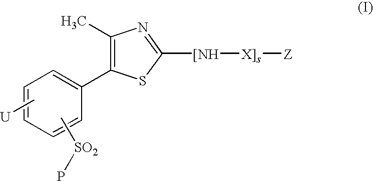
wherein:
s is 0 or 1;
U is hydrogen or halogen;
X is —(C═O), an optionally substituted divalent phenylene, pyridinylene, pyrimidinylene, or pyrazinylene radical, or a bond;
P is optionally substituted C₁-C₆ alkyl and Z is —(CH₂)_{z}—X₁-L₁-NHCHR₁R₂; or Z is optionally substituted C₁-C₆ alkyl and P is —(CH₂)_{z}—X₁-L₁-NHCHR₁R₂;
R₁ is a carboxylic acid group (—COOH), or an ester group which is hydrolysable by one or more intracellular carboxylesterase enzymes to a carboxylic acid group;
R₂ is the side chain of a natural or non-natural alpha amino acid;
X₁ is (i) a bond; —NR₄C(═O)NR₅— or —NR₄S(═O)₂—; or except when X is —(C═O)— (ii) —C(═O)—, —S(═O)₂—, or —S(═O)₂NR₄— wherein R₄ and R₅ are independently hydrogen or optionally substituted C₁-C₆ alkyl;
z is 0 or 1;
L₁ represents a divalent radical of formula -(Alk¹)ₘ(O)ₙ(Alk²)ₚ— wherein m, n and p are independently 0 or 1,
Q is (i) an optionally substituted divalent mono- or bicyclic carbocyclic or heterocyclic radical having 5-13 ring members, or (ii), in the case where both m and p are 0, a divalent radical of formula —X²-Q¹- or -Q¹-X²- wherein X² is —O—, —S— or —NR^{A}— wherein R^{A} is hydrogen or optionally substituted C₁-C₃ alkyl, and Q^{I} is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl.

2. A compound as claimed in claim 1 of formula (IA) or a salt, N-oxide, hydrate or solvate thereof:

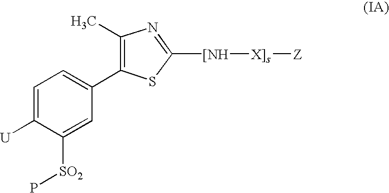

3. A compound as claimed in claim 1 wherein U is chloro.

4. A compound as claimed in claim 1 wherein P is methyl.

5. A compound as claimed in claim 1 wherein X is —(C═O)—.

6. A compound as claimed in claim 5 wherein X₁ is a bond.

7. A compound as claimed in claim 1 wherein X is 1,3-phenylene, 1,4-phenylene, or one of the following divalent radicals:

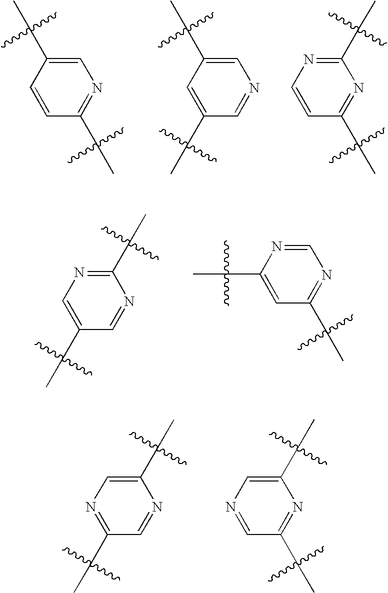

8. A compound as claimed in claim 1 wherein X is a bond.

9. A compound as claimed in claim 1 wherein z is 0.

10. A compound as claimed in claim 1 wherein, in the radical L₁, Alk¹ and Alk², when present, are selected from —CH₂—, CH₂CH₂—, CH₂CH₂CCH₂—, and divalent cyclopropyl, cyclopentyl and cyclohexyl radicals.

11. A compound as claimed in claim 1 wherein, in the radical L₁, Q when present is 1,4-phenylene.

12. A compound as claimed in claim 1 wherein, in the radical L¹, m and p are 0.

13. A compound as claimed in claim 1 wherein, in the radical L¹, n and p are 0 and m is 1.

14. A compound as claimed in any of claim 1 wherein, in the radical L¹, m, n and p are all 0.

15. A compound as claimed in claim 1 wherein X is —(C═O)— and the radical -L₁-X₁—[CH₂]_{z}—, is —CH₂—, —CH₂CH₂—, —CH₂CH₂CH₂—, or —CH₂CH₂CH₂CH₂—.

16. A compound as claimed in claim 1 wherein R₁ is a carboxylic acid group.

17. A compound as claimed in claim 1 wherein R₁ is an ester group of formula —(C═O)OR₇ wherein R₇ is R₈R₉R₁₀C— wherein
(i) R₈ is hydrogen or optionally substituted (C₁-C₃)alkyl-(Z¹)ₐ-[(C₁-C₃)alkyl]_{b}— or (C₂-C₃)alkenyl-(Z¹)ₐ-[(C₁-C₃)alkyl]_{b}— wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6- ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen.

18. A compound as claimed in claim 17 wherein R₁₀ is hydrogen.

19. A compound as claimed in claim 17 wherein R₇ is methyl, ethyl, n- or iso-propyl, n-, sec-, or tert-butyl, cyclohexyl, allyl, phenyl, benzyl, 2-, 3- or 4-pyridylmethyl, N-methylpiperidin-4-yl, tetrahydrofuran-3-yl or methoxyethyl.

20. A compound as claimed in claim 17 wherein R₇ is cyclopentyl.

21. A compound as claimed in claim 1 wherein R₂ is hydrogen.

22. A compound as claimed in claim 1 wherein R₂ is phenyl, benzyl, cyclohexyl or iso-butyl.

23. A compound as claimed in claim 1 wherein R₁ is an ester group of formula —(C═O)OR₇ wherein R₇ is cyclopentyl, and R₂ is hydrogen, phenyl, benzyl, or iso-butyl.

24. A compound as claimed in claim 1 which has formula (IE): 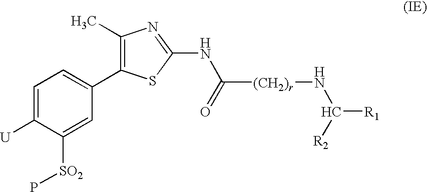
wherein U is chloro, P is methyl, R₁ is a carboxylic acid group or an ester group of formula —(C═O)OR₇ wherein R₇ is R₈R₉R₁₀C— wherein
(i) R₈ is hydrogen or optionally substituted (C₁-C₃)alkyl-(Z¹)ₐ-[(C₁-C₃)alkyl]_{b}- or (C₂-C₃)alkenyl-(Z¹)ₐ-[(C₁-C₃)alkyl]_{b}- wherein a and b are independently 0 or 1 and Z¹ is O—, —S— or —NR₁₁ wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6- ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen, and
R₂ is hydrogen, phenyl, benzyl, cyclohexyl or iso-butyl.

25. A compound as claimed in claim 1 which has formula (IF): 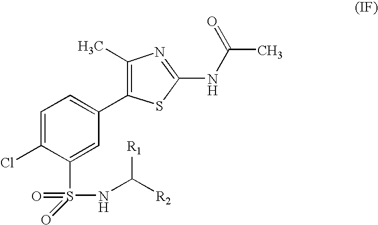
wherein R₁ is a carboxylic acid group or an ester group of formula —(C═O)OR₇ wherein R₇ is R₈R₉R₁₀C— wherein
(i) R₈ is hydrogen or optionally substituted (C₁-C₃)alkyl-(Z¹)ₐ-[(C₁-C₃)alkyl]_{b}- or (C₂-C₃)alkenyl-(Z¹)ₐ-[(C₁-C₃)alkyl]_{b}- wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6- ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen, and
R₂ is hydrogen, phenyl, benzyl cyclohexyl or iso-butyl.

26. A compound as claimed in claim 1 having the structure of any of the compounds of the specific Examples herein.

27. A pharmaceutical composition comprising a compound as claimed in claim 1, together with a pharmaceutically acceptable carrier.

28. (canceled)

29. (canceled)

30. A method of inhibiting the activity of a PI3 kinase enzyme comprising contacting the enzyme with an amount of a compound as claimed in claim 1 effective for such inhibition.

31. A method as claimed in claim 30 for the inhibition of PI3 kinase α and/or PI3 kinase γ activity, ex vivo or in vivo.

32. A method for the treatment of neoplastic, immune or inflammatory disease, which comprises administering to a subject suffering such disease an effective amount of a compound as claimed in claim 1.

33. The method as claimed in claim 30 for the treatment of cancer cell proliferation.

34. The method as claimed in claim 30 for the treatment of cancers.

35. The method as claimed in claim 30 for the treatment of rheumatoid arthritis, psoriasis, inflammatory bowel disease, Crohn's disease, ulcerative colitis, chronic obstructive pulmonary disease, asthma, multiple sclerosis, diabetes, atopic dermatitis, graft versus host disease, or systemic lupus erythematosus.

36. The method as claimed in claim 34 wherein the cancers are selected from bowel cancer, ovarian cancer, head and neck and cervical squamous cancers, gastric and lung cancers, anaplastic oligodendrogliomas, glioblastoma multiforme or medulloblastomas
